SEQUENCE LISTING (32 sequences)

SEQ ID NO: 1 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - DNA, 726 bp, Homo sapiens
  source 1..726: mol_type = genomic DNA
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 3 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 4 - DNA, 585 bp, Homo sapiens
  source 1..585: mol_type = genomic DNA
atgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaa

SEQ ID NO: 5 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 6 - DNA, 462 bp, Homo sapiens
  source 1..462: mol_type = genomic DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 7 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 8 - DNA, 2187 bp, Homo sapiens
  source 1..2187: mol_type = genomic DNA
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 9 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 10 - DNA, 483 bp, Homo sapiens
  source 1..483: mol_type = genomic DNA
atggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctga

SEQ ID NO: 11 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 12 - DNA, 1173 bp, Homo sapiens
  source 1..1173: mol_type = genomic DNA
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 13 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 14 - DNA, 1329 bp, Homo sapiens
  source 1..1329: mol_type = genomic DNA
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 15 - protein, 309 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 16 - DNA, 930 bp, Homo sapiens
  source 1..930: mol_type = genomic DNA
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 17 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 18 - DNA, 1542 bp, Homo sapiens
  source 1..1542: mol_type = genomic DNA
atgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaa

SEQ ID NO: 19 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 20 - DNA, 3624 bp, Homo sapiens
  source 1..3624: mol_type = genomic DNA
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtga

SEQ ID NO: 21 - protein, 210 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGC

SEQ ID NO: 22 - DNA, 636 bp, Homo sapiens
  source 1..636: mol_type = genomic DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 23 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 24 - DNA, 603 bp, Homo sapiens
  source 1..603: mol_type = genomic DNA
atggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtag

SEQ ID NO: 25 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 26 - DNA, 1239 bp, Homo sapiens
  source 1..1239: mol_type = genomic DNA
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 27 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 28 - DNA, 2796 bp, Homo sapiens
  source 1..2796: mol_type = genomic DNA
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactatgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggttagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtagagacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcaggtgagaattttaaagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 29 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 30 - DNA, 1257 bp, Homo sapiens
  source 1..1257: mol_type = genomic DNA
atgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaa

SEQ ID NO: 31 - protein, 751 aa, Homo sapiens
MAFRTICVLVGVFICSICVKGSSQPQARVYLTFDELRETKTSEYFSLSHHPLDYRILLMDEDQDRIYVGSKDHILSLNINNISQEALSVFWPASTIKVEECKMAGKDPTHGCGNFVRVIQTFNRTHLYVCGSGAFSPVCTYLNRGRRSEDQVFMIDSKCESGKGRCSFNPNVNTVSVMINEELFSGMYIDFMGTDAAIFRSLTKRNAVRTDQHNSKWLSEPMFVDAHVIPDGTDPNDAKVYFFFKEKLTDNNRSTKQIHSMIARICPNDTGGLRSLVNKWTTFLKARLVCSVTDEDGPETHFDELEDVFLLETDNPRTTLVYGIFTTSSSVFKGSAVCVYHLSDIQTVFNGPFAHKEGPNHQLISYQGRIPYPRPGTCPGGAFTPNMRTTKEFPDDVVTFIRNHPLMYNSIYPIHKRPLIVRIGTDYKYTKIAVDRVNAADGRYHVLFLGTDRGTVQKVVVLPTNNSVSGELILEELEVFKNHAPITTMKISSKKQQLYVSSNEGVSQVSLHRCHIYGTACADCCLARDPYCAWDGHSCSRFYPTGKRRSRRQDVRHGNPLTQCRGFNLKAYRNAAEIVQYGVKNNTTFLECAPKSPQASIKWLLQKDKDRRKEVKLNERIIATSQGLLIRSVQGSDQGLYHCIATENSFKQTIAKINFKVLDSEMVAVVTDKWSPWTWASSVRALPFHPKDIMGAFSHSEMQMINQYCKDTRQQHQQGDESQKMRGDYGKLKALINSRKSRNRRNQLPES

SEQ ID NO: 32 - DNA, 2256 bp, Homo sapiens
  source 1..2256: mol_type = genomic DNA
atggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtatacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaggtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtccagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccatggacctgggccagctctgtgagggctttacccttccacccgaaggacatcatgggggcattcagccactcagaaatgcagatgattaaccaatattgcaaagacactcggcagcaacatcagcagggagatgaatcacagaaaatgagaggggactatggcaagttaaaggccctcatcaatagtcggaaaagtagaaacaggaggaatcagttgccagagtcataa

(19) United States
(12) Patent Application Publication
(10) Pub. No.: US 2025/0235492 A1
(43) Pub. Date: Jul. 24, 2025

(54) METHODS AND PHARMACEUTICAL COMPOSITIONS FOR MODULATING TEAR FILM COMPOSITION TO TREAT CORNEAL DEFECTS

(71) Applicants: INSTITUT NATIONAL DE LA SANTÉ ET DE LA RECHERCHE MÉDICALE, Paris (FR); UNIVERSITÉ DE MONTPELLIER, Montpellier (FR); CENTRE HOSPITALIER UNIVERSITAIRE DE MONTPELLIER, Montpellier (FR)

(72) Inventors: Frédéric MICHON, Montpellier (FR); Vincent DAIEN, Montpellier (FR); Benoit GAUTIER, Montpellier (FR)

(21) Appl. No.: 18/832,633

(22) PCT Filed: Feb. 15, 2023
(86) PCT No.: PCT/EP2023/053810

§ 371 Date: Jul. 24, 2024

(30) Foreign Application Priority Data
Feb. 15, 2022 (EP) 22305168.1

(51) Int. Cl. A61K 35/76; A61K 38/18; A61K 48/00; A61P 27/02; C07K 14/48; C12N 15/86

(52) U.S. Cl. CPC A61K 35/76; A61K 38/185; A61K 48/0075; A61P 27/02; C07K 14/48; C12N 15/86

(57) ABSTRACT

Tears are a complex biological fluid secreted by the lacrimal apparatus. Their composition can change according to the context. After a corneal wound, the lacrimal gland secretes reflex tears, which contain specific growth factors supporting the wound healing process. In specific pathological contexts, such as dry eye disease, the tear composition is not able to support corneal regeneration, nor basic homeostasis. In this study, the inventors used an AAV2/9-mediated gene transfer to supplement the tear film. They show that a single AAV2/9 CAG-m-NGF injection in the lacrimal gland neither modify the lacrimal gland physiology, nor alters corneal integrity. Furthermore, NGF is detected for several months in the tear film. Finally, we demonstrate that the secreted NGF is functionally active as it improve the reinnervation of the cornea, following an epithelial abrasion. Altogether, their findings provide a new clinically applicable approach to heal corneal pathologies.

### FIELD OF THE INVENTION

The present invention relates to pharmaceutical field. More particularly, the invention relates to a method for modulating tear film composition in a subject in need thereof.

### BACKGROUND OF THE INVENTION

To ensure clear vision, the anterior parts of the eye, namely the cornea and lens, need to be fully transparent. While the lens is protected by being located inside the eye, the cornea is the most external tissue of the eye, and thus prone to environmental aggressions. This ectodermal organ is subjected to life-long cell renewal, which relies on stem and progenitor cells¹. To coordinate epithelium homeostasis, corneal microenvironment is composed of epithelial cell-cell communication, dense innervation, and tear film. The latter is the source of corneal hydration and nutrients for the epithelium². Moreover, after wounding, the tear composition changes to support corneal wound healing, through a modification of the factors secreted by the lacrimal gland (LG)³. The tear composition change requires an efficient sensory network of the cornea. The dense corneal innervation is essential to maintain corneal physiology. First, blinking and tear composition adaptation depends on the activity of corneal sensory nerves. Furthermore, the neurotrophic factors released by the nerves for the epithelium are crucial for homeostasis and wound healing⁴.

Among the most prominent causes for corneal defects, three are making most of the influx of patients in hospital. First cause, physical wounds, such as abrasions⁵, are mostly caused by small foreign objects that scratch the epithelium. This injury is painful, and the subsequent oedema provokes photophobia and impairs visual acuity. If the particle gets embedded within the epithelium, corneal irregularities might form, resulting in continuous pain and significant visual disruption⁶. Dry eye diseases account for the second cause of corneal defects. Dry eye diseases can arise from genetic disease, such as Gougerot-Sjörgren syndrome⁷, or from ageing, as up to ⅓ of the elder population can be affected^{8,9}. The altered tear film has an imbalanced composition, offering less nutrients and growth factors to the corneal epithelium, which in turn affects corneal homeostasis. Consequently, persistent epithelial defects appear, such as ulceration, melting and perforation, impairing sight¹⁰. Third cause, neurotrophic keratitis is due to a partial or complete loss of corneal innervation, causing a defected corneal homeostasis¹¹. Neurotrophic keratitis results from defected corneal wound healing after abrasion or transplant, from neurodegenerative diseases, or from chronic metabolic diseases, such as diabetes. Currently 415 million adults globally are diagnosed with diabetes, and the World Health Organization projected that there will be 640 million adults by 2040¹². While being underdiagnosed, diabetic keratopathy affects 47 to 64% of diabetic adults¹³. The main symptom of neurotrophic keratitis is corneal ulceration and perforation.

The current treatments for these corneal defects are topical and consist in eye drops, which can be supplemented with autologous serum¹⁴, or nerve growth factor (NGF) in the case of neurotrophic keratitis¹⁵. Not only these treatments are heavy for patients, especially autologous serum, but the frequent lack of patient compliance to a prescribed eyedrop regimen can lead to increased sight defects¹⁶. Their costs are also a burden for the society. There is thus a need to identify novel treatment for these corneal defects.

These treatments solely rely on the addition of an external eye drop solution mimicking the tear film composition without considering the LG which produces, secretes, and modulates the tear film composition. Consequently, using LG directly as a bioreactor would constitute an appealing alternative strategy to modulate the tear film composition. This could be achieved by adenovirus-associated virus (AAV) vector-mediated gene transfer into LG. Indeed, AAV vectors present many advantages for gene delivery. They efficiently transduce a broad range of cells in which they allow long-lasting transgene expression. Importantly, they trigger limited/mild immunogenic responses in vivo which overall asserts their biosafety¹⁷. Numerous AAV-based gene therapies have emerged as evidenced by many ongoing clinical trials, namely for neurodegenerative, neuromuscular, cardiovascular, ocular genetic diseases and cancer^{18,19}. Only three AAV-based gene therapies have been approved by the FDA, among which two are still used. Luxturna® (voretigene neparvovec) is used for the treatment of biallelic RPE65 mutation-associated retinal dystrophy²⁰ while Zolgensma® (onasemnogene abeparvovec-xioi) is delivered to paediatric patients under two years of age suffering from spinal muscular atrophy²¹.

In this study, we present a novel strategy to modulate corneal physiology through targeted tear film modification by transferring a gene of interest into LG.

The inventor herein present a novel strategy to rescue corneal physiology through targeted tear film modification by transferring a chosen gene in the lacrimal gland.

### SUMMARY OF THE INVENTION

In a first aspect, the present invention relates to methods and pharmaceutical compositions for modulating tear film composition in a subject in need thereof, comprising administering into the lacrimal gland of said subject a vector comprising a nucleic acid encoding a protein of interest. The methods of the invention are particularly suitable to treat corneal defects in subject in need thereof.

In particular, the present invention is defined by the claims.

### DETAILED DESCRIPTION OF THE INVENTION

The inventors confirm that AAV-mediated gene transfer was feasible in murine LG using AAV2/5 or AAV2/9. After demonstrating that all epithelial cell types are prone to AAV-mediated gene transfer, they used AAV2/5-mNGF and AAV2/9-mNGF, as proof of concept, to establish the parameters for efficient gene transfer, allowing a targeted modification of tear composition. They investigated the impact of AAV2 serotype impact on secreted protein level and chose AAV2/9-mNGF to investigate the duration of tear film modulation, as well as the safety of such an approach on the cornea. They show that a single AAV2/9 CAG-mNGF injection in the lacrimal gland does increase the volume of tears. Altogether, their results demonstrate that a single AAV2/9 injection into murine LG could be used to specifically modify the tear film and consequently to support corneal physiology in pathological contexts, such as neurotrophic keratitis, or recurring abscesses. The same kind of targeted tear film modification is obtained after injecting mRNA into murine lacrimal gland. They show that a single injection of mRNA encoding GFP or mNGF is enough to lead to the protein secretion within the tear film for a short period of time.

Their findings provide a new clinically applicable approach to heal corneal pathologies.

### Therapeutic Methods and Uses

Accordingly, in a first aspect the present invention relates to a method for modulating tear film composition in a subject in need thereof, comprising administering into the lacrimal gland of said subject a nucleic acid encoding a protein of interest.

As used herein, the term “tear film”, has its general meaning in the art and refers to a thin fluid layer covering the cornea and acting as barrier between the eye and environment. This film is transparent and has an aqueous/mucin phase, decreasing in mucin concentration towards a distinct superficial lipid layer. The outermost layer of the tear film is composed predominantly of lipids and is referred to as the Tear Film Lipid Layer (TFLL). The most significant role of the lipid layer is in retarding evaporation of tears from the ocular surface. Most of the aqueous fluid is secreted from the lacrimal glands, which also secretes a specific variety of proteins, electrolytes, and water (Dartt D. A et al, 2013).

Alterations of tear lipids composition and properties are related to dry eye syndrome. After wounding, the tear composition changes to support corneal wound healing, through a modification of the factors secreted by the lacrimal gland (Kuony, 2019). The tear composition change requires an efficient sensory network of the cornea. The dense corneal innervation is essential to maintain corneal physiology.

As used herein, the term “subject” refers to any mammals, such as a human, a rodent, a feline, a canine, and a primate. Particularly, in the present invention, the subject is a human afflicted with or susceptible to be afflicted with corneal defects. Particularly, in the present invention, the subject is a human afflicted with or susceptible to be afflicted with disease associated with corneal defects. Particularly, in the present invention, the subject is a human afflicted with or susceptible to be afflicted with physical wounds corneal, dry eye disease, diabetic keratopathy, corneal thinning disorders such as keratoconus, ICE syndrome or neurotrophic keratitis (NK). Particularly, in the present invention, the subject is a human afflicted with or susceptible to be afflicted with drye eye diseases. Particularly, in the present invention, the subject is a human on which an eye surgery (such as corneal transplantation) has been performed or will be performed.

As used herein, the term “nucleic acid” has its general meaning in the art and refers to a deoxyribonucleic acid (DNA) or a ribonucleic acid (RNA).

DNA is a polymer composed of two polynucleotide chains that coil around each other to form a double helix. The polymer carries genetic instructions for the development, functioning, growth and reproduction of all known organisms and many viruses. Each strand has a backbone made of alternating sugar (deoxyribose) and phosphate groups. Attached to each sugar is one of four bases: adenine (A), cytosine (C), guanine (G) or thymine (T). RNA is a single-stranded biopolymer which consists of ribose nucleotides (nitrogenous bases appended to a ribose sugar) attached by phosphodiester bonds, forming strands of varying lengths. The nitrogenous bases in RNA are adenine, guanine, cytosine, and uracil, which replaces thymine in DNA. RNA includes messenger RNA (mRNA), transfer RNA (tRNA), and ribosomal RNA. mRNA refers to RNA made from a DNA template during the process of transcription. mRNA is read by a ribosome in the process of synthesizing a protein.

In particular embodiment, the nucleic acid is a messenger RNA (mRNA).

Nucleid acid (DNA or RNA) as used herein encompasses both modified and unmodified nucleic acid. DNA or RNA (such mRNA) may contain one or more coding and non-coding regions. DNA or RNA (such as mRNA) can be purified from natural sources, produced using recombinant expression systems and optionally purified, chemically synthesized, etc. Where appropriate, e.g., in the case of chemically synthesized molecules, DNA mRNA can comprise nucleoside analogs such as analogs having chemically modified bases or sugars, backbone modifications, etc. An nucleic acide sequence is presented in the 5′ to 3′ direction unless otherwise indicated. In some embodiments, the mRNA comprises natural nucleosides {e.g., adenosine, guanosine, cytidine, uridine); nucleoside analogs {e.g., 2-aminoadenosine, 2-thiothymidine, inosine, pyrrolo-pyrimidine, 3-methyl adenosine, 5-methylcytidine, C-5 propynyl-cytidine, C-5 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, and 2-thiocytidine); chemically modified bases; biologically modified bases {e.g., methylated bases); intercalated bases; modified sugars {e.g., 2′-fluororibose, ribose, 2′-deoxyribose, arabinose, and hexose); and/or modified phosphate groups {e.g., phosphorothioates and 5′-N-phosphoramidite linkages). In some embodiments, the DNA comprises natural nucleosides {e.g., adenosine, guanosine, cytidine, thymine), 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil,-uracil-5-oxyacetic acid methylester, uracil-5-oxyacetic acid, pseudouracil, queosine, 2-thiocytosine, and 2,6-diaminopurine.

As used herein, the term “lacrimal gland” (LG) has its general meaning in the art and refers to exocrine gland that secret the aqueous layer of the tear film onto the surface of the conjunctiva and cornea of the eye. In some embodiments, the nucleic acid encoding a protein of interest, in particular the messenger ribonucleic acid (mRNA) is administered into the lacrimal gland which has been previously anesthetized. In some embodiments, the vector is administered into the lacrimal gland which has been previously anesthetized.

In some embodiment, according to all the methods of the invention, the protein of interest is a growth factor. According to the invention, the term “protein of interest” includes natural and recombinant analog of the protein of interest.

As used herein, the term “growth factor” has its general meaning in the art, and refers to a naturally occurring substance capable of stimulating cell proliferation, wound healing, and occasionally cellular differentiation. In some embodiment, the protein of interest is a growth factor promoting healing of corneal. According to the invention, the term “growth factor” includes natural and recombinant analog of the growth factor.

Growth factors promoting healing of corneal are very well known in the art and include adrenomedullin, angiopoietin, vascular Endothelial Growth Factor (VEGF) such as VEGF-A, epidermal growth factor (EGF), ciliary neurotrophic factor (CNTF), autocrine motility factor, ephrins, hepatocyte growth factor (HGF), neuropilin 2 (NRP2), semaphorin (SEMA) such as semaphorin 3C (SEMA3C), pigment epithelium-derived factor (PEDF) also known as serpin F1 (SERPINF1), thyroglobuline, insulin, insulin-like growth factors (IGF) such as insulin-like growth factor 1 (IGF-1) and insulin-like growth factor 2 (IGF-2), interleukins, neuregulins, neurotrophins such as nerve growth factor (NGF), brain-derived neutrophic factor (BDNF), neurothropin-3 (NT-3) and neurotrophin-4 (NT-4), platelet-derived growth factor (PDGF), transforming growth factor such as transforming growth factor alpha (TGF-α) and transforming growth factor beta (TGF-β), tumor necrosis factor-alpha (TNF-α), glial-cell derived neurotrophic factor (GDNF), fibroblast growth factor (FGF), such as fibroblast growth factor 7 (FGF-7) also know as keratinocyte growth factor (KGF), and bone morphogenesis protein (BMP), such as bone morphogenesis protein 6 (BMP-6).

In some embodiment, according to all the methods of the invention, the protein of interest is selected in the group consisting of nerve growth factor (NGF), vascular Endothelial Growth Factor A (VEGF-A), epidermal growth factor (EGF), brain-derived neutrophic factor (BDNF), ciliary neurotrophic factor (CNTF), keratinocyte growth factor (KGF), hepatocyte growth factor (HGF), neuropilin 2 (NRP2), semaphorin 3C SEMA3C, pigment epithelium-derived factor (PEDF) also known as serpin F1 (SERPINF1), thyroglobuline, insulin-like growth factor 1 (IGF-1), insulin-like growth factor 2 (IGF-2), neurothropin-3 (NT-3), neurothropin-4 (NT-4), tumor necrosis factor-alpha (TNF-α), insulin, bone morphogenesis protein 6 (BMP-6), glial-cell derived neurotrophic factor (GDNF) and transforming growth factor (TGF).

In some embodiment, according to all the methods of the invention, the protein of interest is selected in the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), insulin-like growth factor 2 (IGF-2), glial-cell derived neurotrophic factor (GDNF) and transforming growth factor (TGF).

As used the term “nerve growth factor” (NGF) has its general meaning in the art and refers to a neurotrophic factor and neuropeptide primarily involved in the regulation of growth, proliferation of nerve cells. The NGF gene is located in chromosome 1. Its Entrez reference is 4803. Its Unit Prot is P01138. Exemplary amino acid sequences of NGF include sequences a set forth in SEQ ID NO:1. Exemplary acid nucleic coding for the nerve growth factor is NM_002506. In particular embodiment, the nucleic acid encoding NGF comprises an amino acid sequence having at least 90% of identity with the sequence as set forth in SEQ ID NO:2. In particular embodiment, the nucleic acid encoding NGF comprises or consists of SEQ ID NO: 2. In particular embodiment, the nerve growth factor is a recombinant human nerve growth factor. In particular embodiment the nerve growth factor is cernergermin.

According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

SEQ ID NO: 1: amino acid sequence of the human NGF
MSMLFYTLITAFLIGIQAEPHSESNVPAGHTIPQAHWTKLQHSLDTALRRARSA
PAAAIAARVAGQTRNITVDPRLFKKRRLRSPRVLFSTQPPREAADTQDLDFEVGGAAP
FNRTHRSKRSSSHPIFHRGEFSVCDSVSVWVGDKTTATDIKGKEVMVLGEVNINNSVF
KQYFFETKCRDPNPVDSGCRGIDSKHWNSYCTTTHTFVKALTMDGKQAAWRFIRIDT
ACVCVLSRKAVRRA
SEQ ID NO: 2: nucleic acid sequence coding for human NGF
atgtccatgttgttctacactctgatcacagcttttctgatcggcatacaggggaaccacactcagagagcaatgtccctgca
ggacacaccatcccccaagcccactggactaaacttcagcattcccttgacactgcccttcgcagagcccgcagcgccccggcagcg
gcgatagctgcacgcgtggcggggcagacccgcaacattactgtggaccccaggctgtttaaaaagcggcgactccgttcaccccgt
gtgctgtttagcacccagcctccccgtgaagctgcagacactcaggatctggacttcgaggtcggtggtgctgcccccttcaacaggac
tcacaggagcaagcggtcatcatcccatcccatcttccacaggggcgaattctcggtgtgtgacagtgtcagcgtgtgggttggggata
agaccaccgccacagacatcaagggcaaggaggtgatggtgttgggagaggtgaacattaacaacagtgtattcaaacagtactttttt
gagaccaagtgccgggacccaaatcccgttgacagcgggtgccggggcattgactcaaagcactggaactcatattgtaccacgact
cacacctttgtcaaggcgctgaccatggatggcaagcaggctgcctggcggtttatccggatagatacggcctgtgtgtgtgtgctcag
caggaaggctgtgagaagagcctga

As used the term “fibroblast growth factor” (FGF), has its general meaning in the art and refers to a growth factor produced by macrophages; they are involved in a wide variety of processes, most notably as crucial elements for normal development in animal cells. Any irregularities in their function lead to a range of developmental defects. FGF family include FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23. In particular, the FGF is FGF-7 (or KGF). In particular embodiment, the fibroblast growth factor is a recombinant human fibroblast growth factor.

As used the term “keratinocyte growth factor” (KGF), also known as FGF7, has its general meaning in the art and refers to a growth factor present in the epithelialization-phase of wound healing that binds to fibroblast growth factor receptor 2b (FGFR2b). The FGF7 gene is located in chromosome 15. Its Entrez reference is 2252. Its Unit Prot is P21781. Exemplary acid nucleic coding for the keratinocyte growth factor is NM_002009. Exemplary amino acid sequences of NGF include sequences a set forth in SEQ ID NO:3. In particular embodiment, the nucleic acid encoding KGF comprises an amino acid sequence having at least 90% of identity with the sequence as set forth in SEQ ID NO:4. In particular embodiment, the nucleic acid encoding KGF comprises or consists of SEQ ID NO:4. In particular embodiment, the keratinocyte growth factor is a recombinant human fibroblast growth factor.

SEQ ID NO: 3: amino acid sequence of the human KGF
MHKWILTWILPTLLYRSCFHIICLVGTISLACNDMTPEQMATNVNCSSPERHTR
SYDYMEGGDIRVRRLFCRTQWYLRIDKRGKVKGTQEMKNNYNIMEIRTVAVGIVAI
KGVESEFYLAMNKEGKLYAKKECNEDCNFKELILENHYNTYASAKWTHNGGEMFV
ALNQKGIPVRGKKTKKEQKTAHFLPMAIT
SEQ ID NO: 4 nucleic acid sequence coding for human KGF
atgcacaaatggatactgacatggatcctgccaactttgctctacagatcatgctttcacattatctgtctagtgggtactatatct
ttagcttgcaatgacatgactccagagcaaatggctacaaatgtgaactgttccagccctgagcgacacacaagaagttatgattacatg
gaaggaggggatataagagtgagaagactcttctgtcgaacacagtggtacctgaggatcgataaaagaggcaaagtaaaagggac
ccaagagatgaagaataattacaatatcatggaaatcaggacagtggcagttggaattgtggcaatcaaaggggtggaaagtgaattct
atcttgcaatgaacaaggaaggaaaactctatgcaaagaaagaatgcaatgaagattgtaacttcaaagaactaattctggaaaaccatt
acaacacatatgcatcagctaaatggacacacaacggaggggaaatgtttgttgccttaaatcaaaaggggattcctgtaagaggaaaa
aaaacgaagaaagaacaaaaaacagcccactttcttcctatggcaataacttaa

As used the term “insulin-like growth factor” (IGF-1), also known as somatomedin C, has its general meaning in the art and refers to a hormone similar to insulin. The IGF1 gene is located in chromosome 12. Its Entrez reference is 3479. Its Unit Prot is P05019. Exemplary of acid nucleic coding for the insulin-like growth factor is NM_000618, NM_001111283, NM 001111284 or NM_001111285. Exemplary amino acid sequences of IGF-1 include sequences a set forth in SEQ ID NO:5. In particular embodiment, the nucleic acid encoding IGF-1 comprises an amino acid sequence having at least 90% of identity with the sequence as set forth in SEQ ID NO:6. In particular embodiment, the nucleic acid encoding IGF-1 comprises or consists of SEQ ID NO:6. In particular embodiment, the insulin-like growth factor is a recombinant human insulin-like growth factor.

SEQ ID NO: 5: amino acid sequence of the human IGF-1
MGKISSLPTQLFKCCFCDFLKVKMHTMSSSHLFYLALCLLTFTSSATAGPETLC
GAELVDALQFVCGDRGFYFNKPTGYGSSSRRAPQTGIVDECCFRSCDLRRLEMYCAP
LKPAKSARSVRAQRHTDMPKTQKEVHLKNASRGSAGNKNYRM
SEQ ID NO: 6: nucleic acid sequence coding for human IGF-1
atgggaaaaatcagcagtcttccaacccaattatttaagtgctgcttttgtgatttcttgaaggtgaagatgcacaccatgtcctc
ctcgcatctcttctacctggcgctgtgcctgctcaccttcaccagctctgccacggctggaccggagacgctctgcggggctgagctgg
tggatgctcttcagttcgtgtgtggagacaggggcttttatttcaacaagcccacagggtatggctccagcagtcggagggcgcctcag
acaggcatcgtggatgagtgctgcttccggagctgtgatctaaggaggctggagatgtattgcgcacccctcaagcctgccaagtcag
ctcgctctgtccgtgcccagcgccacaccgacatgcccaagacccagaaggaagtacatttgaagaacgcaagtagagggagtgca
ggaaacaagaactacaggatgtag

As used the term “hepatocyte growth factor” (HGF), also known as scatter factor (SF) has its general meaning in the art and refers to a paracrine cellular growth, motility and morphogenic factor secreted by mesenchymal cells. The HGF gene is located in chromosome 7. Its Entrez reference is 3082. Its Unit Prot is P14210. Exemplary acid nucleic coding for the hepatocyte growth factor is NM_000601, NM_001010931, NM_001010932, NM_001010933 or NM_001010934. Exemplary amino acid sequences of HGF include sequences a set forth in SEQ ID NO:7. In particular embodiment, the nucleic acid encoding HGF comprises an amino acid sequence having at least 90% of identity with the sequence as set forth in SEQ ID NO:8. In particular embodiment, the nucleic acid encoding HGF comprises or consists of SEQ ID NO: 8. In particular embodiment, the hepatocye growth factor is a recombinant human hepatocyte growth factor.

SEQ ID NO: 7: amino acid sequence of the human HGF
MWVTKLLPALLLQHVLLHLLLLPIAIPYAEGQRKRRNTIHEFKKSAKTTLIKID
PALKIKTKKVNTADQCANRCTRNKGLPFTCKAFVFDKARKQCLWFPFNSMSSGVKK
EFGHEFDLYENKDYIRNCIIGKGRSYKGTVSITKSGIKCQPWSSMIPHEHSFLPSSYRG
KDLQENYCRNPRGEEGGPWCFTSNPEVRYEVCDIPQCSEVECMTCNGESYRGLMDH
TESGKICQRWDHQTPHRHKFLPERYPDKGFDDNYCRNPDGQPRPWCYTLDPHTRWE
YCAIKTCADNTMNDTDVPLETTECIQGQGEGYRGTVNTIWNGIPCQRWDSQYPHEH
DMTPENFKCKDLRENYCRNPDGSESPWCFTTDPNIRVGYCSQIPNCDMSHGQDCYRG
NGKNYMGNLSQTRSGLTCSMWDKNMEDLHRHIFWEPDASKLNENYCRNPDDDAH
GPWCYTGNPLIPWDYCPISRCEGDTTPTIVNLDHPVISCAKTKQLRVVNGIPTRTNIG
WMVSLRYRNKHICGGSLIKESWVLTARQCFPSRDLKDYEAWLGIHDVHGRGDEKCK
QVLNVSQLVYGPEGSDLVLMKLARPAVLDDFVSTIDLPNYGCTIPEKTSCSVYGWGY
TGLINYDGLLRVAHLYIMGNEKCSQHHRGKVTLNESEICAGAEKIGSGPCEGDYGGP
LVCEQHKMRMVLGVIVPGRGCAIPNRPGIFVRVAYYAKWIHKIILTYKVPQS
SEQ ID NO: 8: nucleic acid sequence coding for human HGF
atgtgggtgaccaaactcctgccagccctgctgctgcagcatgtcctcctgcatctcctcctgctccccatcgccatcccctat
gcagagggacaaaggaaaagaagaaatacaattcatgaattcaaaaaatcagcaaagactaccctaatcaaaatagatccagcactga
agataaaaaccaaaaaagtgaatactgcagaccaatgtgctaatagatgtactaggaataaaggacttccattcacttgcaaggcttttgt
ttttgataaagcaagaaaacaatgcctctggttccccttcaatagcatgtcaagtggagtgaaaaaagaatttggccatgaatttgacctct
atgaaaacaaagactacattagaaactgcatcattggtaaaggacgcagctacaagggaacagtatctatcactaagagtggcatcaaa
tgtcagccctggagttccatgataccacacgaacacagctttttgccttcgagctatcggggtaaagacctacaggaaaactactgtcga
aatcctcgaggggaagaagggggaccctggtgtttcacaagcaatccagaggtacgctacgaagtctgtgacattcctcagtgttcag
aagttgaatgcatgacctgcaatggggagagttatcgaggtctcatggatcatacagaatcaggcaagatttgtcagcgctgggatcatc
agacaccacaccggcacaaattcttgcctgaaagatatcccgacaagggctttgatgataattattgccgcaatcccgatggccagccg
aggccatggtgctatactcttgaccctcacacccgctgggagtactgtgcaattaaaacatgcgctgacaatactatgaatgacactgat
gttcctttggaaacaactgaatgcatccaaggtcaaggagaaggctacaggggcactgtcaataccatttggaatggaattccatgtcag
cgttgggattctcagtatcctcacgagcatgacatgactcctgaaaatttcaagtgcaaggacctacgagaaaattactgccgaaatcca
gatgggtctgaatcaccctggtgttttaccactgatccaaacatccgagttggctactgctcccaaattccaaactgtgatatgtcacatgg
acaagattgttatcgtgggaatggcaaaaattatatgggcaacttatcccaaacaagatctggactaacatgttcaatgtgggacaagaa
catggaagacttacatcgtcatatcttctgggaaccagatgcaagtaagctgaatgagaattactgccgaaatccagatgatgatgctcat
ggaccctggtgctacacgggaaatccactcattccttgggattattgccctatttctcgttgtgaaggtgataccacacctacaatagtcaa
tttagaccatcccgtaatatcttgtgccaaaacgaaacaattgcgagttgtaaatgggattccaacacgaacaaacataggatggatggtt
agtttgagatacagaaataaacatatctgcggaggatcattgataaaggagagttgggttcttactgcacgacagtgtttcccttctcgag
acttgaaagattatgaagcttggcttggaattcatgatgtccacggaagaggagatgagaaatgcaaacaggttctcaatgtttcccagct
ggtatatggccctgaaggatcagatctggttttaatgaagcttgccaggcctgctgtcctggatgattttgttagtacgattgatttaccta
attatggatgcacaattcctgaaaagaccagttgcagtgtttatggctggggctacactggattgatcaactatgatggcctattacgagtg
gcacatctctatataatgggaaatgagaaatgcagccagcatcatcgagggaaggtgactctgaatgagtctgaaatatgtgctggggct
gaaaagattggatcaggaccatgtgagggggattatggtggcccacttgtttgtgagcaacataaaatgagaatggttcttggtgtcattg
ttcctggtcgtggatgtgccattccaaatcgtcctggtatttttgtccgagtagcatattatgcaaaatggatacacaaaattattttaacat
ataaggtaccacagtcatag

As used the term “transforming growth factor” (TGF), also known as “tumor growth factor” has its general meaning in the art and refers to a family of polypeptide growth factors. In particular embodiment, the transforming growth factor is a recombinant human transforming growth factor. TGF include TGFα and TGFβ. Transforming growth factor alpha (TGF-α) is a protein that in humans is encoded by the TGFA gene, located in chromosome 2. Its Entrez reference is 7039. Exemplary acid nucleic coding for the TGFα is NM_001099691, NM_001308158, NM_001308159 or NM_003236. Its Uniprot reference is P01135. Exemplary amino acid sequences of TGF-α include sequences a set forth in SEQ ID NO:9. In particular embodiment, the nucleic acid encoding TGF-α comprises an amino acid sequence having at least 90% of identity with the sequence as set forth in SEQ ID NO: 10. In particular embodiment, the nucleic acid encoding TGF-α comprises or consists of SEQ ID NO:10. In particular embodiment, the transforming growth factor α is a recombinant human transforming growth factor α. Transforming growth factor beta (TGF-β) is a multifunctional cytokine that include three different mammalian isoforms (TGF-β1, TGF-β2 and TGF-β3). TGF-β1 is encoded by the TGFB1 gene. Its Entrez reference is 7040. Exemplary acid nucleic coding for the TGF-β1 is NM_000660. Its Uniprot reference is P01137. Exemplary amino acid sequences of TGF-β1 include sequences a set forth in SEQ ID NO:11. In particular embodiment, the nucleic acid encoding TGF-β1 comprises an amino acid sequence having at least 90% of identity with the sequence as set forth in SEQ ID NO:12. In particular embodiment, the nucleic acid encoding TGF-β1 comprises or consists of SEQ ID NO:12. In particular embodiment, the transforming growth factor β1 is a recombinant human transforming growth factor β1. TGF-β2 is encoded by the TGFβ2 gene. Its Entrez reference is 7042. Exemplary acid nucleic coding for the TGF-β2 is NM_003238 or NM_001135599. In particular embodiment, the transforming growth factor β2 is a recombinant human transforming growth factor β2. Exemplary amino acid sequences of TGF-β2 include sequences a set forth in SEQ ID NO:13. In particular embodiment, the nucleic acid encoding TGF-β2 comprises an amino acid sequence having at least 90% of identity with the sequence as set forth in SEQ ID NO: 14. In particular embodiment, the nucleic acid encoding TGF-β2 comprises or consists of SEQ ID NO: 14. TGF-β3 is encoded by the TGFB3 gene. Its Entrez reference is 7043. Exemplary acid nucleic coding for the TGF-β2 is NM_003239, NM_001329938 or NM_001329939. Its Uniprot reference is P61812. Exemplary amino acid sequences of TGF-β2 include sequences a set forth in SEQ ID NO:15. In particular embodiment, the DNA encoding TGF-β2 comprises an amino acid sequence having at least 90% of identity with the sequence as set forth in SEQ ID NO:16. In particular embodiment, the nucleic acid encoding TGF-β2 comprises or consists of SEQ ID NO:16. In particular embodiment, the transforming growth factor β3 is a recombinant human transforming growth factor β3.

SEQ ID NO: 9: amino acid sequence of the human TGF-α
MVPSAGQLALFALGIVLAACQALENSTSPLSADPPVAAAVVSHFNDCPDSHTQ
FCFHGTCRFLVQEDKPACVCHSGYVGARCEHADLLAVVAASQKKQAITALVVVSIV
ALAVLIITCVLIHCCQVRKHCEWCRALICRHEKPSALLKGRTACCHSETVV
SEQ ID NO: 10: nucleic acid sequence coding for human TGF-α
Atggtcccctcggctggacagctcgccctgttcgctctgggtattgtgttggctgcgtgccaggccttggagaacagcacgt
ccccgctgagtgcagacccgcccgtggctgcagcagtggtgtcccattttaatgactgcccagattcccacactcagttctgcttccatg
gaacctgcaggtttttggtgcaggaggacaagccagcatgtgtctgccattctgggtacgttggtgcacgctgtgagcatgcggacctc
ctggccgtggtggctgccagccagaagaagcaggccatcaccgccttggtggtggtctccatcgtggccctggctgtccttatcatcac
atgtgtgctgatacactgctgccaggtccgaaaacactgtgagtggtgccgggccctcatctgccggcacgagaagcccagcgccct
cctgaagggaagaaccgcttgctgccactcagaaacagtggtctga
SEQ ID NO: 11: amino acid sequence of the human TGF-β1
MPPSGLRLLPLLLPLLWLLVLTPGRPAAGLSTCKTIDMELVKRKRIEAIRGQILS
KLRLASPPSQGEVPPGPLPEAVLALYNSTRDRVAGESAEPEPEPEADYYAKEVTRVL
MVETHNEIYDKFKQSTHSIYMFFNTSELREAVPEPVLLSRAELRLLRLKLKVEQHVEL
YQKYSNNSWRYLSNRLLAPSDSPEWLSFDVTGVVRQWLSRGGEIEGFRLSAHCSCDS
RDNTLQVDINGFTTGRRGDLATIHGMNRPFLLLMATPLERAQHLQSSRHRRALDTNY
CFSSTEKNCCVRQLYIDFRKDLGWKWIHEPKGYHANFCLGPCPYIWSLDTQYSKVLA
LYNQHNPGASAAPCCVPQALEPLPIVYYVGRKPKVEQLSNMIVRSCKCS
SEQ ID NO: 12: nucleic acid sequence coding for human TGF-β1
atgccgccctccgggctgcggctgctgccgctgctgctaccgctgctgtggctactggtgctgacgcctggccggccggc
cgcgggactatccacctgcaagactatcgacatggagctggtgaagcggaagcgcatcgaggccatccgcggccagatcctgtcca
agctgcggctcgccagccccccgagccagggggaggtgccgcccggcccgctgcccgaggccgtgctcgccctgtacaacagca
cccgcgaccgggtggccggggagagtgcagaaccggagcccgagcctgaggccgactactacgccaaggaggtcacccgcgtg
ctaatggtggaaacccacaacgaaatctatgacaagttcaagcagagtacacacagcatatatatgttcttcaacacatcagagctccga
gaagcggtacctgaacccgtgttgctctcccgggcagagctgcgtctgctgaggctcaagttaaaagtggagcagcacgtggagctgt
accagaaatacagcaacaattcctggcgatacctcagcaaccggctgctggcacccagcgactcgccagagtggttatcttttgatgtc
accggagttgtgcggcagtggttgagccgtggaggggaaattgagggctttcgccttagcgcccactgctcctgtgacagcagggata
acacactgcaagtggacatcaacgggttcactaccggccgccgaggtgacctggccaccattcatggcatgaaccggcctttcctgctt
ctcatggccaccccgctggagagggcccagcatctgcaaagctcccggcaccgccgagccctggacaccaactattgcttcagctcc
acggagaagaactgctgcgtgcggcagctgtacattgacttccgcaaggacctcggctggaagtggatccacgagcccaagggcta
ccatgccaacttctgcctcgggccctgcccctacatttggagcctggacacgcagtacagcaaggtcctggccctgtacaaccagcat
aacccgggcgcctcggcggcgccgtgctgcgtgccgcaggcgctggagccgctgcccatcgtgtactacgtgggccgcaagccca
aggtggagcagctgtccaacatgatcgtgcgctcctgcaagtgcagctga
SEQ ID NO: 13: amino acid sequence of the human TGF-β2
MHYCVLSAFLILHLVTVALSLSTCSTLDMDQFMRKRIEAIRGQILSKLKLTSPP
EDYPEPEEVPPEVISIYNSTRDLLQEKASRRAAACERERSDEEYYAKEVYKIDMPPFFP
SETVCPVVTTPSGSVGSLCSRQSQVLCGYLDAIPPTFYRPYFRIVRFDVSAMEKNASN
LVKAEFRVFRLQNPKARVPEQRIELYQILKSKDLTSPTQRYIDSKVVKTRAEGEWLSF
DVTDAVHEWLHHKDRNLGFKISLHCPCCTFVPSNNYIIPNKSEELEARFAGIDGTSTY
TSGDQKTIKSTRKKNSGKTPHLLLMLLPSYRLESQQTNRRKKRALDAAYCFRNVQDN
CCLRPLYIDFKRDLGWKWIHEPKGYNANFCAGACPYLWSSDTQHSRVLSLYNTINPE
ASASPCCVSQDLEPLTILYYIGKTPKIEQLSNMIVKSCKCS
SEQ ID NO: 14: nucleic acid sequence coding for human TGF-β2
atgcactactgtgtgctgagcgcttttctgatcctgcatctggtcacggtcgcgctcagcctgtctacctgcagcacactcgat
atggaccagttcatgcgcaagaggatcgaggcgatccgcgggcagatcctgagcaagctgaagctcaccagtcccccagaagacta
tcctgagcccgaggaagtccccccggaggtgatttccatctacaacagcaccagggacttgctccaggagaaggcgagccggagg
cggccgcctgcgagcgcgagaggagcgacgaagagtactacgccaaggaggtttacaaaatagacatgccgcccttcttcccctcc
gaaactgtctgcccagttgttacaacaccctctggctcagtgggcagcttgtgctccagacagtcccaggtgctctgtgggtaccttgat
gccatcccgcccactttctacagaccctacttcagaattgttcgatttgacgtctcagcaatggagaagaatgcttccaatttggtgaaagc
agagttcagagtctttcgtttgcagaacccaaaagccagagtgcctgaacaacggattgagctatatcagattctcaagtccaaagattta
acatctccaacccagcgctacatcgacagcaaagttgtgaaaacaagagcagaaggcgaatggctctccttcgatgtaactgatgctgt
tcatgaatggcttcaccataaagacaggaacctgggatttaaaataagcttacactgtccctgctgcacttttgtaccatctaataattacat
catcccaaataaaagtgaagaactagaagcaagatttgcaggtattgatggcacctccacatataccagtggtgatcagaaaactataaa
gtccactaggaaaaaaaacagtgggaagaccccacatctcctgctaatgttattgccctcctacagacttgagtcacaacagaccaacc
ggcggaagaagcgtgctttggatgcggcctattgctttagaaatgtgcaggataattgctgcctacgtccactttacattgatttcaagag
ggatctagggtggaaatggatacacgaacccaaagggtacaatgccaacttctgtgctggagcatgcccgtatttatggagttcagaca
ctcagcacagcagggtcctgagcttatataataccataaatccagaagcatctgcttctccttgctgcgtgtcccaagatttagaacctcta
accattctctactacattggcaaaacacccaagattgaacagctttctaatatgattgtaaagtcttgcaaatgcagctaa
SEQ ID NO: 15: amino acid sequence of the human TGF-β3
MKMHLQRALVVLALLNFATVSLSLSTCTTLDFGHIKKKRVEAIRGQILSKLRL
TSPPEPTVMTHVPYQVLALYNSTRELLEEMHGEREEGCTQENTESEYYAKEIHKFDMI
QGLAEHNELAVCPKGITSKVFRFNVSSVEKNRTNLFRAEFRVLRVPNPSSKRNEQRIE
LFQILRPDEHIAKQRYIGGKNLPTRGTAEWLSFDVTDTVREWLLRRESNLGLEISIHCP
CHTFQPNGDILENIHEVMEIKFKGVDNEDDHGRGDLGRLKKQKDHHNPHLILMMIPP
HRLDNPGQGGQRKKRALDTNYCFR
SEQ ID NO: 16: nucleic acid sequence of the human TGF-β3
atgaagatgcacttgcaaagggctctggtggtcctggccctgctgaactttgccacggtcagcctctctctgtccacttgcacc
accttggacttcggccacatcaagaagaagagggtggaagccattaggggacagatcttgagcaagctcaggctcaccagcccccct
gagccaacggtgatgacccacgtcccctatcaggtcctggccctttacaacagcacccgggagctgctggaggagatgcatgggga
gagggaggaaggctgcacccaggaaaacaccgagtcggaatactatgccaaagaaatccataaattcgacatgatccaggggctgg
cggagcacaacgaactggctgtctgccctaaaggaattacctccaaggttttccgcttcaatgtgtcctcagtggagaaaaatagaacca
acctattccgagcagaattccgggtcttgcgggtgcccaaccccagctctaagcggaatgagcagaggatcgagctcttccagatcctt
cggccagatgagcacattgccaaacagcgctatatcggtggcaagaatctgcccacacggggcactgccgagtggctgtcctttgatg
tcactgacactgtgcgtgagtggctgttgagaagagagtccaacttaggtctagaaatcagcattcactgtccatgtcacacctttcagcc
caatggagatatcctggaaaacattcacgaggtgatggaaatcaaattcaaaggcgtggacaatgaggatgaccatggccgtggagat
ctggggcgcctcaagaagcagaaggatcaccacaaccctcatctaatcctcatgatgattcccccacaccggctcgacaacccgggc
caggggggtcagaggaagaagcgggctttggacaccaattactgcttccggtga

As used the term “bone morphogenesis protein” (BMP) has its general meaning in the art and refers to a group of growth factors also known as cytokines and as metabologens. BMPs interact with specific receptors on the cell surface, referred to as bone morphogenetic protein receptors (BMPRs). BMP family include BMP1, BMP2, BMP3, BMP4, BMP5, BMP6, BMP7, BMP8a, BMP8b, BMP10, BMP11 and BMP15. In particular, the BMP is BMP-6.

As used the term “Bone morphogenetic protein 6” (BMP-6), also known as VGR, has its general meaning in the art and refers to the key regulator of hepcidin, the small peptide secreted by the liver which is the major regulator of iron metabolism in mammals. Its Entrez reference is 654. Exemplary acid nucleic coding for the keratinocyte growth factor is NM_001718. Its Uniprot reference is P22004. Exemplary amino acid sequences of BMP-6 include sequences a set forth in SEQ ID NO:17. In particular embodiment, the nucleic acid encoding BMP-6 comprises an amino acid sequence having at least 90% of identity with the sequence as set forth in SEQ ID NO:18. In particular embodiment, the nucleic acid encoding BMP-6 comprises or consists of SEQ ID NO:18. In particular embodiment, the bone morphogenetic protein 6 is a recombinant human bone morphogenetic protein 6.

SEQ ID NO: 17: amino acid sequence of the human BMP-6
MPGLGRRAQWLCWWWGLLCSCCGPPPLRPPLPAAAAAAAGGQLLGDGGSP
GRTEQPPPSPQSSSGFLYRRLKTQEKREMQKEILSVLGLPHRPRPLHGLQQPQPPALRQ
QEEQQQQQQLPRGEPPPGRLKSAPLFMLDLYNALSADNDEDGASEGERQQSWPHEA
ASSSQRRQPPPGAAHPLNRKSLLAPGSGSGGASPLTSAQDSAFLNDADMVMSFVNLV
EYDKEFSPRQRHHKEFKFNLSQIPEGEVVTAAEFRIYKDCVMGSFKNQTFLISIYQVLQ
EHQHRDSDLFLLDTRVVWASEEGWLEFDITATSNLWVVTPQHNMGLQLSVVTRDGV
HVHPRAAGLVGRDGPYDKQPFMVAFFKVSEVHVRTTRSASSRRRQQSRNRSTQSQD
VARVSSASDYNSSELKTACRKHELYVSFQDLGWQDWIIAPKGYAANYCDGECSFPL
NAHMNATNHAIVQTLVHLMNPEYVPKPCCAPTKLNAISVLYFDDNSNVILKKYRNM
VVRACGCH
SEQ ID NO: 18: amino acid sequence of the human BMP-6
atgccggggctggggcggagggcgcagtggctgtgctggtggtgggggctgctgtgcagctgctgcgggcccccgccg
ctgcggccgcccttgcccgctgccgcggccgccgccgccggggggcagctgctgggggacggcgggagccccggccgcacgg
agcagccgccgccgtcgccgcagtcctcctcgggcttcctgtaccggcggctcaagacgcaggagaagcgggagatgcagaagga
gatcttgtcggtgctggggctcccgcaccggccccggcccctgcacggcctccaacagccgcagcccccggcgctccggcagcag
gaggagcagcagcagcagcagcagctgcctcgcggagagccccctcccgggcgactgaagtccgcgcccctcttcatgctggatct
gtacaacgccctgtccgccgacaacgacgaggacggggcgtcggagggggagaggcagcagtcctggccccacgaagcagcca
gctcgtcccagcgtcggcagccgcccccgggcgccgcgcacccgctcaaccgcaagagccttctggcccccggatctggcagcgg
cggcgcgtccccactgaccagcgcgcaggacagcgccttcctcaacgacgcggacatggtcatgagctttgtgaacctggtggagta
cgacaaggagttctcccctcgtcagcgacaccacaaagagttcaagttcaacttatcccagattcctgagggtgaggtggtgacggctg
cagaattccgcatctacaaggactgtgttatggggagttttaaaaaccaaacttttcttatcagcatttatcaagtcttacaggagcatca
gcacagagactctgacctgtttttgttggacacccgtgtagtatgggcctcagaagaaggctggctggaatttgacatcacggccactagc
aatctgtgggttgtgactccacagcataacatggggcttcagctgagcgtggtgacaagggatggagtccacgtccacccccgagccg
caggcctggtgggcagagacggcccttacgacaagcagcccttcatggtggctttcttcaaagtgagtgaggtgcacgtgcgcacca
ccaggtcagcctccagccggcgccgacaacagagtcgtaatcgctctacccagtcccaggacgtggcgcgggtctccagtgcttcag
attacaacagcagtgaattgaaaacagcctgcaggaagcatgagctgtatgtgagtttccaagacctgggatggcaggactggatcatt
gcacccaagggctatgctgccaattactgtgatggagaatgctccttcccactcaacgcacacatgaatgcaaccaaccacgcgattgt
gcagaccttggttcaccttatgaaccccgagtatgtccccaaaccgtgctgtgcgccaactaagctaaatgccatctcggttctttacttt
gatgacaactccaatgtcattctgaaaaaatacaggaatatggttgtaagagcttgtggatgccactaa

As used the term “epidermal growth factor” (EGF) has its general meaning in the art and refers to a protein that stimulates cell growth and differentiation by binding to its receptor, EGFR. The EGF gene is located in chromosome 4. Its Entrez reference is 1950. Exemplary acid nucleic coding for the epidermal growth factor is NM_001178130, NM_001178131, NM_001963 and NM_001357021. Its Uniprot reference is P01133. Exemplary amino acid sequences of EGF include sequences a set forth in SEQ ID NO:19. In particular embodiment, the DNA encoding EGF comprises an amino acid sequence having at least 90% of identity with the sequence as set forth in SEQ ID NO:20. In particular embodiment, the nucleic acid encoding EGF comprises or consists of SEQ ID NO:20. In particular embodiment, the epidermal growth factor is a recombinant human epidermal growth factor.

In some embodiments, the methods according to the invention, wherein the vector is a non-viract vector when the acid nucleic encodes for EGF.

SEQ ID NO: 19: amino acid sequence of the human EGF
MLLTLIILLPVVSKFSFVSLSAPQHWSCPEGTLAGNGNSTCVGPAPFLIFSHGNS
IFRIDTEGTNYEQLVVDAGVSVIMDFHYNEKRIYWVDLERQLLQRVFLNGSRQERVC
NIEKNVSGMAINWINEEVIWSNQQEGIITVTDMKGNNSHILLSALKYPANVAVDPVER
FIFWSSEVAGSLYRADLDGVGVKALLETSEKITAVSLDVLDKRLFWIQYNREGSNSLI
CSCDYDGGSVHISKHPTQHNLFAMSLFGDRIFYSTWKMKTIWIANKHTGKDMVRINL
HSSFVPLGELKVVHPLAQPKAEDDTWEPEQKLCKLRKGNCSSTVCGQDLQSHLCMC
AEGYALSRDRKYCEDVNECAFWNHGCTLGCKNTPGSYYCTCPVGFVLLPDGKRCH
QLVSCPRNVSECSHDCVLTSEGPLCFCPEGSVLERDGKTCSGCSSPDNGGCSQLCVPL
SPVSWECDCFPGYDLQLDEKSCAASGPQPFLLFANSQDIRHMHFDGTDYGTLLSQQM
GMVYALDHDPVENKIYFAHTALKWIERANMDGSQRERLIEEGVDVPEGLAVDWIGR
RFYWTDRGKSLIGRSDLNGKRSKIITKENISQPRGIAVHPMAKRLFWTDTGINPRIESS
SLQGLGRLVIASSDLIWPSGITIDFLTDKLYWCDAKQSVIEMANLDGSKRRRLTQNDV
GHPFAVAVFEDYVWFSDWAMPSVMRVNKRTGKDRVRLQGSMLKPSSLVVVHPLAK
PGADPCLYQNGGCEHICKKRLGTAWCSCREGFMKASDGKTCLALDGHQLLAGGEV
DLKNQVTPLDILSKTRVSEDNITESQHMLVAEIMVSDQDDCAPVGCSMYARCISEGE
DATCQCLKGFAGDGKLCSDIDECEMGVPVCPPASSKCINTEGGYVCRCSEGYQGDGI
HCLDIDECQLGEHSCGENASCTNTEGGYTCMCAGRLSEPGLICPDSTPPPHLREDDHH
YSVRNSDSECPLSHDGYCLHDGVCMYIEALDKYACNCVVGYIGERCQYRDLKWWE
LRHAGHGQQQKVIVVAVCVVVLVMLLLLSLWGAHYYRTQKLLSKNPKNPYEESSR
DVRSRRPADTEDGMSSCPQPWFVVIKEHQDLKNGGQPVAGEDGQAADGSMQPTSW
RQEPQLCGMGTEQGCWIPVSSDKGSCPQVMERSFHMPSYGTQTLEGGVEKPHSLLSA
NPLWQQRALDPPHQMELTQ
SEQ ID NO: 20: nucleic acid sequence of the human EGF
atgctgctcactcttatcattctgttgccagtagtttcaaaatttagttttgttagtctctcagcaccgcagcactggagctgtcctg
aaggtactctcgcaggaaatgggaattctacttgtgtgggtcctgcacccttcttaattttctcccatggaaatagtatctttaggattgac
acagaaggaaccaattatgagcaattggtggtggatgctggtgtctcagtgatcatggattttcattataatgagaaaagaatctattgggt
ggatttagaaagacaacttttgcaaagagtttttctgaatgggtcaaggcaagagagagtatgtaatatagagaaaaatgtttctggaatggc
aataaattggataaatgaagaagttatttggtcaaatcaacaggaaggaatcattacagtaacagatatgaaaggaaataattcccacatt
cttttaagtgctttaaaatatcctgcaaatgtagcagttgatccagtagaaaggtttatattttggtcttcagaggtggctggaagcctttat
agagcagatctcgatggtgtgggagtgaaggctctgttggagacatcagagaaaataacagctgtgtcattggatgtgcttgataagcggc
tgttttggattcagtacaacagagaaggaagcaattctcttatttgctcctgtgattatgatggaggttctgtccacattagtaaacatccaa
cacagcataatttgtttgcaatgtccctttttggtgaccgtatcttctattcaacatggaaaatgaagacaatttggatagccaacaaacaca
ctggaaaggacatggttagaattaacctccattcatcatttgtaccacttggtgaactgaaagtagtgcatccacttgcacaacccaaggca
gaagatgacacttgggagcctgagcagaaactttgcaaattgaggaaaggaaactgcagcagcactgtgtgtgggcaagacctccag
tcacacttgtgcatgtgtgcagagggatacgccctaagtcgagaccggaagtactgtgaagatgttaatgaatgtgctttttggaatcatg
gctgtactcttgggtgtaaaaacacccctggatcctattactgcacgtgccctgtaggatttgttctgcttcctgatgggaaacgatgtcatc
aacttgtttcctgtccacgcaatgtgtctgaatgcagccatgactgtgttctgacatcagaaggtcccttatgtttctgtcctgaaggctcag
tgcttgagagagatgggaaaacatgtagcggttgttcctcacccgataatggtggatgtagccagctctgcgttcctcttagcccagtatc
ctgggaatgtgattgctttcctgggtatgacctacaactggatgaaaaaagctgtgcagcttcaggaccacaaccatttttgctgtttgcca
attctcaagatattcgacacatgcattttgatggaacagactatggaactctgctcagccagcagatgggaatggtttatgccctagatcat
gaccctgtggaaaataagatatactttgcccatacagccctgaagtggatagagagagctaatatggatggttcccagcgagaaaggct
tattgaggaaggagtagatgtgccagaaggtcttgctgtggactggattggccgtagattctattggacagacagagggaaatctctgat
tggaaggagtgatttaaatgggaaacgttccaaaataatcactaaggagaacatctctcaaccacgaggaattgctgttcatccaatggc
caagagattattctggactgatacagggattaatccacgaattgaaagttcttccctccaaggccttggccgtctggttatagccagctctg
atctaatctggcccagtggaataacgattgacttcttaactgacaagttgtactggtgcgatgccaagcagtctgtgattgaaatggccaat
ctggatggttcaaaacgccgaagacttacccagaatgatgtaggtcacccatttgctgtagcagtgtttgaggattatgtgtggttctcaga
ttgggctatgccatcagtaatgagagtaaacaagaggactggcaaagatagagtacgtctccaaggcagcatgctgaagccctcatca
ctggttgtggttcatccattggcaaaaccaggagcagatccctgcttatatcaaaacggaggctgtgaacatatttgcaaaaagaggctt
ggaactgcttggtgttcgtgtcgtgaaggttttatgaaagcctcagatgggaaaacgtgtctggctctggatggtcatcagctgttggcag
gtggtgaagttgatctaaagaaccaagtaacaccattggacatcttgtccaagactagagtgtcagaagataacattacagaatctcaac
acatgctagtggctgaaatcatggtgtcagatcaagatgactgtgctcctgtgggatgcagcatgtatgctcggtgtatttcagagggag
aggatgccacatgtcagtgtttgaaaggatttgctggggatggaaaactatgttctgatatagatgaatgtgagatgggtgtcccagtgtg
cccccctgcctcctccaagtgcatcaacaccgaaggtggttatgtctgccggtgctcagaaggctaccaaggagatgggattcactgtc
ttgatattgatgagtgccaactgggggagcacagctgtggagagaatgccagctgcacaaatacagagggaggctatacctgcatgtg
tgctggacgcctgtctgaaccaggactgatttgccctgactctactccaccccctcacctcagggaagatgaccaccactattccgtaag
aaatagtgactctgaatgtcccctgtcccacgatgggtactgcctccatgatggtgtgtgcatgtatattgaagcattggacaagtatgcat
gcaactgtgttgttggctacatcggggagcgatgtcagtaccgagacctgaagtggtgggaactgcgccacgctggccacgggcagc
agcagaaggtcatcgtggtggctgtctgcgtggtggtgcttgtcatgctgctcctcctgagcctgtggggggcccactactacaggact
cagaagctgctatcgaaaaacccaaagaatccttatgaggagtcgagcagagatgtgaggagtcgcaggcctgctgacactgaggat
gggatgtcctcttgccctcaaccttggtttgtggttataaaagaacaccaagacctcaagaatgggggtcaaccagtggctggtgaggat
ggccaggcagcagatgggtcaatgcaaccaacttcatggaggcaggagccccagttatgtggaatgggcacagagcaaggctgctg
gattccagtatccagtgataagggctcctgtccccaggtaatggagcgaagctttcatatgccctcctatgggacacagacccttgaagg
gggtgtcgagaagccccattctctcctatcagctaacccattatggcaacaaagggccctggacccaccacaccaaatggagctgact
cagtga

As used the term “glial-cell derived neutrophic factor” (GDNF) has its general meaning in the art and refers to a small protein that potently promotes the survival of many types of neurons. It signals through GFRα receptors, particularly GFRα1.Its Entrez reference is 2668. Exemplary acid nucleic coding for the glial-cell derived neutrophic factor is NM_000514, NM_001190468, NM_001190469, NM_001278098 and NM_199231. Its Uniprot reference is P39905. Exemplary amino acid sequences of GDNF include sequences a set forth in SEQ ID NO: 21. In particular embodiment, the nucleic acid encoding GDNF comprises an amino acid sequence having at least 90% of identity with the sequence as set forth in SEQ ID NO:22. In particular embodiment, the nucleic acid encoding GDNF comprises or consists of SEQ ID NO: 22. In particular embodiment, the GDNF is a recombinant human GDNF.

SEQ ID NO: 21: amino acid sequence of the human GDNF
MKLWDVVAVCLVLLHTASAFPLPAGKRPPEAPAEDRSLGRRRAPFALSSDSN
MPEDYPDQFDDVMDFIQATIKRLKRSPDKQMAVLPRRERNRQAAAANPENSRGKGR
RGQRGKNRGCVLTAIHLNVTDLGLGYETKEELIFRYCSGSCDAAETTYDKILKNLSR
NRRLVSDKVGQACCRPIAFDDDLSFLDDNLVYHILRKHSAKRCGCI
SEQ ID NO: 22: nucleic acid sequence of the human GDNF
atgaagttatgggatgtcgtggctgtctgcctggtgctgctccacaccgcgtccgccttcccgctgcccgccggtaagaggc
ctcccgaggcgcccgccgaagaccgctccctcggccgccgccgcgcgcccttcgcgctgagcagtgactcaaatatgccagaggat
tatcctgatcagttcgatgatgtcatggattttattcaagccaccattaaaagactgaaaaggtcaccagataaacaaatggcagtgcttcc
tagaagagagcggaatcggcaggctgcagctgccaacccagagaattccagaggaaaaggtcggagaggccagaggggcaaaa
accggggttgtgtcttaactgcaatacatttaaatgtcactgacttgggtctgggctatgaaaccaaggaggaactgatttttaggtactgc
agcggctcttgcgatgcagctgagacaacgtacgacaaaatattgaaaaacttatccagaaatagaaggctggtgagtgacaaagtag
ggcaggcatgttgcagacccatcgcctttgatgatgacctgtcgtttttagatgataacctggtttaccatattctaagaaagcattccgcta
aaaggtgtggatgtatctga

As used the term “ciliary neutrophic factor” (CTNF) has its general meaning in the art and refers to a survival factor for various neuronal cell types. Seems to prevent the degeneration of motor axons after axotomy. Its Entrez reference is 1270. Exemplary acid nucleic coding for the ciliary neutrophic factor is NM_000614. Its Uniprot reference is P26441. Exemplary amino acid sequences of CTNF include sequences a set forth in SEQ ID NO:23. In particular embodiment, the DNA encoding CTNF comprises an amino acid sequence having at least 90% of identity with the sequence as set forth in SEQ ID NO:24. In particular embodiment, the nucleic acid encoding CTNF comprises or consists of SEQ ID NO:24. In particular embodiment, the CTNF is a recombinant human CTNF.

SEQ ID NO: 23: amino acid sequence of the human CTNF
MAFTEHSPLTPHRRDLCSRSIWLARKIRSDLTALTESYVKHQGLNKNINLDSA
DGMPVASTDQWSELTEAERLQENLQAYRTFHVLLARLLEDQQVHFTPTEGDFHQAI
HTLLLQVAAFAYQIEELMILLEYKIPRNEADGMPINVGDGGLFEKKLWGLKVLQELS
QWTVRSIHDLRFISSHQTGIPARGSHYIANNKKM
SEQ ID NO: 24: nucleic acid sequence of the human CTNF
atggctttcacagagcattcaccgctgacccctcaccgtcgggacctctgtagccgctctatctggctagcaaggaagattcg
ttcagacctgactgctcttacggaatcctatgtgaagcatcagggcctgaacaagaacatcaacctggactctgcggatgggatgccag
tggcaagcactgatcagtggagtgagctgaccgaggcagagcgactccaagagaaccttcaagcttatcgtaccttccatgttttgttgg
ccaggctcttagaagaccagcaggtgcattttaccccaaccgaaggtgacttccatcaagctatacatacccttcttctccaagtcgctgc
ctttgcataccagatagaggagttaatgatactcctggaatacaagatcccccgcaatgaggctgatgggatgcctattaatgttggagat
ggtggtctctttgagaagaagctgtggggcctaaaggtgctgcaggagctttcacagtggacagtaaggtccatccatgaccttcgtttc
atttcttctcatcagactgggatcccagcacgtgggagccattatattgctaacaacaagaaaatgtag.

As used the term “vascular endothelial growth factor” (VEGF) has its general meaning in the art and refers to a sub-family of growth factors, the platelet-derived growth factor family of cystine-knot growth factors which stimulates the formation of blood vessels. Seems to prevent the degeneration of motor axons after axotomy. VEGF includes VEGF-A, placenta growth factor (PGF), VEGF-B, VEGF-C and VEGF-D. Vascular endothelial growth factor A (VEGF) induces angiogenesis and vascular hyperpermeability in ocular tissues and is therefore a key therapeutic target for eye conditions in which these processes are dysregulated. Its Entrez reference is 7422. Exemplary acid nucleic coding for the ciliary neutrophic factor is NM_003376, NM_001025366, NM_001025367, NM_001025368, NM_001025369. Its Uniprot reference is P15692. Exemplary amino acid sequences of VEGF-A include sequences a set forth in SEQ ID NO:25. In particular embodiment, the nucleic acid encoding VEGF-A comprises an amino acid sequence having at least 90% of identity with the sequence as set forth in SEQ ID NO:26. In particular embodiment, the nucleic acid encoding VEGF-A comprises or consists of SEQ ID NO:26. In particular embodiment, the VEGF is a recombinant human VEGF.

SEQ ID NO: 25: amino acid sequence of the human VEGF-A
MTDRQTDTAPSPSYHLLPGRRRTVDAAASRGQGPEPAPGGGVEGVGARGVA
LKLFVQLLGCSRFGGAVVRAGEAEPSGAARSASSGREEPQPEEGEEEEEKEEERGPQ
WRLGARKPGSWTGEAAVCADSAPAARAPQALARASGRGGRVARRGAEESGPPHSPS
RRGSASRAGPGRASETMNFLLSWVHWSLALLLYLHHAKWSQAAPMAEGGGQNHHE
VVKFMDVYQRSYCHPIETLVDIFQEYPDEIEYIFKPSCVPLMRCGGCCNDEGLECVPT
EESNITMQIMRIKPHQGQHIGEMSFLQHNKCECRPKKDRARQEKKSVRGKGKGQKR
KRKKSRYKSWSVYVGARCCLMPWSLPGPHPCGPCSERRKHLFVQDPQTCKCSCKNT
DSRCKARQLELNERTCRCDKPRR
SEQ ID NO 26: nucleic acid sequence of the human VEGF-A
ctgacggacagacagacagacaccgcccccagccccagctaccacctcctccccggccggcggcggacagtggacgc
ggcggcgagccgcgggcaggggccggagcccgcgcccggaggcggggtggagggggtcggggctcgcggcgtcgcactgaa
acttttcgtccaacttctgggctgttctcgcttcggaggagccgtggtccgcgcgggggaagccgagccgagcggagccgcgagaa
gtgctagctcgggccgggaggagccgcagccggaggagggggaggaggaagaagagaaggaagaggagagggggccgcagt
ggcgactcggcgctcggaagccgggctcatggacgggtgaggcggcggtgtgcgcagacagtgctccagccgcgcgcgctcccc
aggccctggcccgggcctcgggccggggaggaagagtagctcgccgaggcgccgaggagagcgggccgccccacagcccgag
ccggagagggagcgcgagccgcgccggccccggtcgggcctccgaaaccatgaactttctgctgtcttgggtgcattggagccttgc
cttgctgctctacctccaccatgccaagtggtcccaggctgcacccatggcagaaggaggagggcagaatcatcacgaagtggtgaa
gttcatggatgtctatcagcgcagctactgccatccaatcgagaccctggtggacatcttccaggagtaccctgatgagatcgagtacat
cttcaagccatcctgtgtgcccctgatgcgatgcgggggctgctgcaatgacgagggcctggagtgtgtgcccactgaggagtccaac
atcaccatgcagattatgcggatcaaacctcaccaaggccagcacataggagagatgagcttcctacagcacaacaaatgtgaatgca
gaccaaagaaagatagagcaagacaagaaaaaaaatcagttcgaggaaagggaaaggggcaaaaacgaaagcgcaagaaatccc
ggtataagtcctggagcgtgtacgttggtgcccgctgctgtctaatgccctggagcctccctggcccccatccctgtgggccttgctcag
agcggagaaagcatttgtttgtacaagatccgcagacgtgtaaatgttcctgcaaaaacacagactcgcgttgcaaggcgaggcagctt
gagttaaacgaacgtacttgcagatgtgacaagccgaggcggtga

As used the term “neuropilin 2” (NRP2) has its general meaning in the art and refers to a neuropilin factor that binds to SEMA3C, SEMA3F, VEGF-A, VEGF-C, VEGF-D, TGFβ, integrins and ANGPTL4 to promote downstream signaling pathways. Its Entrez reference is 8828. Exemplary acid nucleic coding for the neuropilin-2 is NM_003872, NM_018534, NM_201264, NM_201266 or NM_201267. Its Uniprot reference is 060462. Exemplary amino acid sequences of NRP2 include sequences a set forth in SEQ ID NO:27. In particular embodiment, the nucleic acid encoding NRP2 comprises an amino acid sequence having at least 90% of identity with the sequence as set forth in SEQ ID NO:28. In particular embodiment, the nucleic acid encoding NRP2 comprises or consists of SEQ ID NO:28. In particular embodiment, the NRP2 is a recombinant human NRP2.

SEQ ID NO: 27: amino acid sequence of the human NRP2
MDMFPLTWVFLALYFSRHQVRGQPDPPCGGRLNSKDAGYITSPGYPQDYPSH
QNCEWIVYAPEPNQKIVLNFNPHFEIEKHDCKYDFIEIRDGDSESADLLGKHCGNIAPP
TIISSGSMLYIKFTSDYARQGAGFSLRYEIFKTGSEDCSKNFTSPNGTIESPGFPEKYPH
NLDCTFTILAKPKMEIILQFLIFDLEHDPLQVGEGDCKYDWLDIWDGIPHVGPLIGKYC
GTKTPSELRSSTGILSLTFHTDMAVAKDGFSARYYLVHQEPLENFQCNVPLGMESGRI
ANEQISASSTYSDGRWTPQQSRLHGDDNGWTPNLDSNKEYLQVDLRFLTMLTAIATQ
GAISRETQNGYYVKSYKLEVSTNGEDWMVYRHGKNHKVFQANNDATEVVLNKLH
APLLTRFVRIRPQTWHSGIALRLELFGCRVTDAPCSNMLGMLSGLIADSQISASSTQEY
LWSPSAARLVSSRSGWFPRIPQAQPGEEWLQVDLGTPKTVKGVIIQGARGGDSITAVE
ARAFVRKFKVSYSLNGKDWEYIQDPRTQQPKLFEGNMHYDTPDIRRFDPIPAQYVRV
YPERWSPAGIGMRLEVLGCDWTDSKPTVETLGPTVKSEETTTPYPTEEEATECGENCS
FEDDKDLQLPSGFNCNFDFLEEPCGWMYDHAKWLRTTWASSSSPNDRTFPDDRNFL
RLQSDSQREGQYARLISPPVHLPRSPVCMEFQYQATGGRGVALQVVREASQESKLLW
VIREDQGGEWKHGRIILPSYDMEYQIVFEGVIGKGRSGEIAIDDIRISTDVPLENCMEPI
SAFAGENFKVDIPEIHEREGYEDEIDDEYEVDWSNSSSATSGSGAPSTDKEKSWLYTL
DPILITIIAMSSLGVLLGATCAGLLLYCTCSYSGLSSRSCTTLENYNFELYDGLKHKVK
MNHQKCCSEA
SEQ ID NO: 28: nucleic acid sequence of the human NRP2
atggatatgtttcctctcacctgggttttcttagccctctacttttcaagacaccaagtgagaggccaaccagacccaccgtgc
ggaggtcgtttgaattccaaagatgctggctatatcacctctcccggttacccccaggactacccctcccaccagaactgcgagtggatt
gtttacgcccccgaacccaaccagaagattgtcctcaacttcaaccctcactttgaaatcgagaagcacgactgcaagtatgactttatcg
agattcgggatggggacagtgaatccgcagacctcctgggcaaacactgtgggaacatcgccccgcccaccatcatctcctcgggct
ccatgctctacatcaagttcacctccgactacgcccggcagggggcaggcttctctctgcgctacgagatcttcaagacaggctctgaa
gattgctcaaaaaacttcacaagccccaacgggaccatcgaatctcctgggtttcctgagaagtatccacacaacttggactgcaccttt
accatcctggccaaacccaagatggagatcatcctgcagttcctgatctttgacctggagcatgaccctttgcaggtgggagaggggg
actgcaagtacgattggctggacatctgggatggcattccacatgttggccccctgattggcaagtactgtgggaccaaaacaccctct
gaacttcgttcatcgacggggatcctctccctgacctttcacacggacatggcggtggccaaggatggcttctctgcgcgttactacctg
gtccaccaagagccactagagaactttcagtgcaatgttcctctgggcatggagtctggccggattgctaatgaacagatcagtgcctca
tctacctactctgatgggaggtggacccctcaacaaagccggctccatggtgatgacaatggctggacccccaacttggattccaacaa
ggagtatctccaggtggacctgcgctttttaaccatgctcacggccatcgcaacacagggagcgatttccagggaaacacagaatggc
tactatgtcaaatcctacaagctggaagtcagcactaatggagaggactggatggtgtaccggcatggcaaaaaccacaaggtatttca
agccaacaacgatgcaactgaggtggttctgaacaagctccacgctccactgctgacaaggtttgttagaatccgccctcagacctggc
actcaggtatcgccctccggctggagctcttcggctgccgggtcacagatgctccctgctccaacatgctggggatgctctcaggcctc
attgcagactcccagatctccgcctcttccacccaggaatacctctggagccccagtgcagcccgcctggttagcagccgctcgggct
ggttccctcgaatccctcaggcccagcccggtgaggagtggcttcaggtagatctgggaacacccaagacagtgaaaggtgtcatcat
ccagggagcccgcggaggagacagtatcactgctgtggaagccagagcatttgtgcgcaagttcaaagtctcctacagcctaaacgg
caaggactgggaatacattcaggaccccaggacccagcagccaaagctgttcgaagggaacatgcactatgacacccctgacatcc
gaaggtttgaccccattccggcacagtatgtgcgggtatacccggagaggtggtcgccggcggggattgggatgcggctggaggtg
ctgggctgtgactggacagactccaagcccacggtagagacgctgggacccactgtgaagagcgaagagacaaccaccccctacc
ccaccgaagaggaggccacagagtgtggggagaactgcagctttgaggatgacaaagatttgcagctcccttcgggattcaattgcaa
cttcgatttcctcgaggagccctgtggttggatgtatgaccatgccaagtggctccggaccacctgggccagcagctccagcccaaac
gaccggacgtttccagatgacaggaatttcttgcggctgcagagtgacagccagagagagggccagtatgcccggctcatcagcccc
cctgtccacctgccccgaagcccggtgtgcatggagttccagtaccaggccacgggcggccgcggggggcgctgcaggtggtgc
gggaagccagccaggagagcaagttgctgtgggtcatccgtgaggaccagggcggcgagtggaagcacgggggatcatcctgc
ccagctacgacatggagtaccagattgtgttcgagggagtgatagggaaaggacgttccggagagattgccattgatgacattcggat
aagcactgatgtcccactggagaactgcatggaacccatctcggcttttgcaggtgagaattttaaagtggacatcccagaaatacatga
gagagaaggatatgaagatgaaattgatgatgaatacgaggtggactggagcaattcttcttctgcaacctcagggtctggcgccccct
cgaccgacaaagaaaagagctggctgtacaccctggatcccatcctcatcaccatcatcgccatgagctcactgggcgtcctcctggg
ggccacctgtgcaggcctcctgctctactgcacctgttcctactcgggcctgagctcccgaagctgcaccacactggagaactacaact
tcgagctctacgatggccttaagcacaaggtcaagatgaaccaccaaaagtgctgctccgaggcatga

As used the term “Pigment epithelium-derived factor (PEDF)”, also known as serpin F1 (SERPINF1)” has its general meaning in the art and refers to a multifunctional secreted protein that has anti-angiogenic, anti-tumorigenic, and neurotrophic functions. Its Entrez reference is 5176. Exemplary acid nucleic coding for the PEDF is NM_002615, NM_001329903, NM_001329904 or NM_001329905. Its Uniprot reference is P36955. Exemplary amino acid sequences of PEDF include sequences a set forth in SEQ ID NO:29. In particular embodiment, the nucleic acid encoding PEDF comprises an amino acid sequence having at least 90% of identity with the sequence as set forth in SEQ ID NO:30. In particular embodiment, the nucleic acid encoding PEFD comprises or consists of SEQ ID NO:30. In particular embodiment, the PEDF is a recombinant human PEDF.

SEQ ID NO: 29: amino acid sequence of the human PEDF
MQALVLLLCIGALLGHSSCQNPASPPEEGSPDPDSTGALVEEEDPFFKVPVNKL
AAAVSNFGYDLYRVRSSTSPTTNVLLSPLSVATALSALSLGAEQRTESIIHRALYYDLI
SSPDIHGTYKELLDTVTAPQKNLKSASRIVFEKKLRIKSSFVAPLEKSYGTRPRVLTGN
PRLDLQEINNWVQAQMKGKLARSTKEIPDEISILLLGVAHFKGQWVTKFDSRKTSLE
DFYLDEERTVRVPMMSDPKAVLRYGLDSDLSCKIAQLPLTGSMSIIFFLPLKVTQNLT
LIEESLTSEFIHDIDRELKTVQAVLTVPKLKLSYEGEVTKSLQEMKLQSLFDSPDFSKIT
GKPIKLTQVEHRAGFEWNEDGAGTTPSPGLQPAHLTFPLDYHLNQPFIFVLRDTDTGA
LLFIGKILDPRGP
SEQ ID NO: 30: nucleic acid sequence of the human PEDF
atgcaggccctggtgctactcctctgcattggagccctcctcgggcacagcagctgccagaaccctgccagccccccgga
ggagggctccccagaccccgacagcacaggggcgctggtggaggaggaggatcctttcttcaaagtccccgtgaacaagctggca
gcggctgtctccaacttcggctatgacctgtaccgggtgcgatccagcacgagccccacgaccaacgtgctcctgtctcctctcagtgt
ggccacggccctctcggccctctcgctgggagcggagcagcgaacagaatccatcattcaccgggctctctactatgacttgatcagc
agcccagacatccatggtacctataaggagctccttgacacggtcactgccccccagaagaacctcaagagtgcctcccggatcgtctt
tgagaagaagctgcgcataaaatccagctttgtggcacctctggaaaagtcatatgggaccaggcccagagtcctgacgggcaaccct
cgcttggacctgcaagagatcaacaactgggtgcaggcgcagatgaaagggaagctcgccaggtccacaaaggaaattcccgatga
gatcagcattctccttctcggtgtggcgcacttcaaggggcagtgggtaacaaagtttgactccagaaagacttccctcgaggatttctac
ttggatgaagagaggaccgtgagggtccccatgatgtcggaccctaaggctgttttacgctatggcttggattcagatctcagctgcaag
attgcccagctgcccttgaccggaagcatgagtatcatcttcttcctgcccctgaaagtgacccagaatttgaccttgatagaggagagc
ctcacctccgagttcattcatgacatagaccgagaactgaagaccgtgcaggcggtcctcactgtccccaagctgaagctgagttatga
aggcgaagtcaccaagtccctgcaggagatgaagctgcaatccttgtttgattcaccagactttagcaagatcacaggcaaacccatca
agctgactcaggtggaacaccgggctggctttgagtggaacgaggatggggcgggaaccacccccagcccagggctgcagcctgc
ccacctcaccttcccgctggactatcaccttaaccagcctttcatcttcgtactgagggacacagacacaggggcccttctcttcattggc
aagattctggaccccaggggcccctaa

As used the term “semaphorin” (SEMA), has its general meaning in the art and refers to a class of secreted and membrane proteins that were originally identified as axonal growth cone guidance molecules. Semaphorin includes SEMA3A, SEMA3B, SEMA3C, SEMA3D, SEMA3E, SEMA3F, SEMA3G, SEMA4A, SEMA4B, SEM4C, SEMA4D, SEMA4F, SEMA4G, SEMASA, SEMA5B, SEMA6A, SEMA6B, SEMA6C, SEMA6D, SEMA7A. In particular embodiment, the SEMA is SEMA3C. Semaphorin-3C is a protein that in humans is encoded by the SEMA3C gene and play a role in corneal epithelial wound closure⁵⁹. Its Entrez reference is 10512. Exemplary acid nucleic coding for the SEMA3C is NM_006379, NM 001350120 or NM_001350121. Its Uniprot reference is Q99985. Exemplary amino acid sequences of SEMA3AC include sequences a set forth in SEQ ID NO:31. In particular embodiment, the nucleic acid encoding SEMA3C comprises an amino acid sequence having at least 90% of identity with the sequence as set forth in SEQ ID NO:32. In particular embodiment, the nucleic acid encoding SEMA3C comprises or consists of SEQ ID NO:32. In particular embodiment, the SEMA3C is a recombinant human SEMA3C.

SEQ ID NO: 31: amino acid sequence of the human SEMA3C
MAFRTICVLVGVFICSICVKGSSQPQARVYLTFDELRETKTSEYFSLSHHPLDY
RILLMDEDQDRIYVGSKDHILSLNINNISQEALSVFWPASTIKVEECKMAGKDPTHGC
GNFVRVIQTFNRTHLYVCGSGAFSPVCTYLNRGRRSEDQVFMIDSKCESGKGRCSFNP
NVNTVSVMINEELFSGMYIDFMGTDAAIFRSLTKRNAVRTDQHNSKWLSEPMFVDA
HVIPDGTDPNDAKVYFFFKEKLTDNNRSTKQIHSMIARICPNDTGGLRSLVNKWTTFL
KARLVCSVTDEDGPETHFDELEDVFLLETDNPRTTLVYGIFTTSSSVFKGSAVCVYHL
SDIQTVFNGPFAHKEGPNHQLISYQGRIPYPRPGTCPGGAFTPNMRTTKEFPDDVVTFI
RNHPLMYNSIYPIHKRPLIVRIGTDYKYTKIAVDRVNAADGRYHVLFLGTDRGTVQK
VVVLPTNNSVSGELILEELEVFKNHAPITTMKISSKKQQLYVSSNEGVSQVSLHRCHIY
GTACADCCLARDPYCAWDGHSCSRFYPTGKRRSRRQDVRHGNPLTQCRGFNLKAY
RNAAEIVQYGVKNNTTFLECAPKSPQASIKWLLQKDKDRRKEVKLNERIIATSQGLLI
RSVQGSDQGLYHCIATENSFKQTIAKINFKVLDSEMVAVVTDKWSPWTWASSVRAL
PFHPKDIMGAFSHSEMQMINQYCKDTRQQHQQGDESQKMRGDYGKLKALINSRKSR
NRRNQLPES
SEQ ID NO: 32: nucleic acid sequence of the human SEMA3C
atggcattccggacaatttgcgtgttggttggagtatttatttgttctatctgtgtgaaaggatcttcccagccccaagcaagagt
ttatttaacatttgatgaacttcgagaaaccaagacctctgaatacttcagcctttcccaccatcctttagactacaggattttattaatgg
atgaagatcaggaccggatatatgtgggaagcaaagatcacattctttccctgaatattaacaatataagtcaagaagctttgagtgttttc
tggccagcatctacaatcaaagttgaagaatgcaaaatggctggcaaagatcccacacacggctgtgggaactttgtccgtgtaattcagac
tttcaatcgcacacatttgtatgtctgtgggagtggcgctttcagtcctgtctgtacttacttgaacagagggaggagatcagaggaccaag
ttttcatgattgactccaagtgtgaatctggaaaaggacgctgctctttcaaccccaacgtgaacacggtgtctgttatgatcaatgaggag
cttttctctggaatgtatatagatttcatggggacagatgctgctatttttcgaagtttaaccaagaggaatgcggtcagaactgatcaacat
aattccaaatggctaagtgaacctatgtttgtagatgcacatgtcatcccagatggtactgatccaaatgatgctaaggtgtacttcttcttc
aaagaaaaactgactgacaataacaggagcacgaaacagattcattccatgattgctcgaatatgtcctaatgacactggtggactgcgta
gccttgtcaacaagtggaccactttcttaaaggcgaggctggtgtgctcggtaacagatgaagacggcccagaaacacactttgatgaa
ttagaggatgtgtttctgctggaaactgataacccgaggacaacactagtgtatggcatttttacaacatcaagctcagttttcaaaggatc
agccgtgtgtgtgtatcatttatctgatatacagactgtgtttaatgggccttttgcccacaaagaagggcccaatcatcagctgatttccta
tcagggcagaattccatatcctcgccctggaacttgtccaggaggagcatttacacccaatatgcgaaccaccaaggagttcccagatg
atgttgtcacttttattcggaaccatcctctcatgtacaattccatctacccaatccacaaaaggcctttgattgttcgtattggcactgact
acaagtatacaaagatagctgtggatcgagtgaacgctgctgatgggagataccatgtcctgtttctcggaacagatcggggtactgtgca
aaaagtggttgttcttcctactaacaactctgtcagtggcgagctcattctggaggagctggaagtctttaagaatcatgctcctataacaa
caatgaaaatttcatctaaaaagcaacagttgtatgtgagttccaatgaaggggtttcccaggtatctctgcaccgctgccacatctatggt
acagcctgtgctgactgctgcctggcgcgggacccttattgcgcctgggatggccattcctgttccagattctacccaactgggaaacg
gaggagccgaagacaagatgtgagacatggaaacccactgactcaatgcagaggatttaatctaaaagcatacagaaatgcagctga
aattgtccagtatggagtaaaaaataacaccacttttctggagtgtgcccccaagtctccgcaggcatctatcaagtggctgttacagaaa
gacaaagacaggaggaaagaggttaagctgaatgaacgaataatagccacttcacagggactcctgatccgctctgttcagggttctg
accaaggactttatcactgcattgctacagaaaatagtttcaagcagaccatagccaagatcaacttcaaagttttagattcagaaatggtg
gctgttgtgacggacaaatggtccccatggacctgggccagctctgtgagggctttacccttccacccgaaggacatcatgggggcatt
cagccactcagaaatgcagatgattaaccaatattgcaaagacactcggcagcaacatcagcagggagatgaatcacagaaaatgag
aggggactatggcaagttaaaggccctcatcaatagtcggaaaagtagaaacaggaggaatcagttgccagagtcataa

In some embodiments, according to all the method of the invention, wherein the acid nucleic encodes for NGF or EGF.

In particular embodiment, according to all the method of the invention, the nucleic acid encoding a protein of interest is administered into the lacrimal gland simultaneously, separately or sequentially with a transfection reagent.

In particular embodiment, according to all the method of the invention, the nucleic acid encoding a protein of interest is further mixed with a transfection reagent prior to the administration into lacrimal gland. In particular embodiment, according to all the method of the invention, the nucleic acid encoding a protein of interest is administered together with a transfection reagent. In particular embodiment, according to all the method of the invention, the messenger RNA encoding a protein of interest is further mixed with a transfection reagent prior to the administration into lacrimal gland. In particular embodiment, according to all the method of the invention, the messenger RNA encoding a protein of interest is administered together with a transfection reagent. In other words, in particular embodiment, according to all the method of the invention, the messenger RNA encoding a protein of interest is administered into the lacrimal gland simultaneously, separately or sequentially with a transfection reagent. In particular embodiment, according to all the method of the invention, the messenger RNA encoding NGF or EGF is administered into the lacrimal gland simultaneously, separately or sequentially with a transfection reagent.

As used herein, the term “administration simultaneously” refers to administration of 2 active ingredients (i.e the nucleic acid encoding a protein of interest and trasnsfection reagents) by the same route and at the same time or at substantially the same time. The term “administration separately” refers to an administration of 2 active ingredients (i.e the nucleic acid encoding a protein of interest and trasnsfection reagents) at the same time or at substantially the same time by different routes. The term “administration sequentially” refers to an administration of 2 active ingredients ((i.e the nucleic acid encoding a protein of interest and trasnsfection reagents) at different times, the administration route being identical or different.

Thus, in a particular embodiment, the present invention relates to a method for modulating tear film composition in a subject in need thereof, comprising administering into the lacrimal gland of said subject a messenger ribonucleic acid (mRNA) encoding a protein of interest simultaneously, separately or sequentially with a transfection reagent.

In a particular embodiment, the present invention relates to a method for modulating tear film composition in a subject in need thereof, comprising administering into the lacrimal gland of said subject a messenger ribonucleic acid (mRNA) encoding NGF or EGF simultaneously, separately or sequentially with a transfection reagent.

As used herein, the term “transfection reagent” has its general meaning in the art and refers to any agents suitable to introduction of nucleic acid into eukaryotic cells by non-viral agents. It is well known in the art many various non viral methods to introduce nucleic acid as described in Yamano S et al, Mol Biotechno.2010⁵², Kumar et al, Cold Spring HArb Protoc. 2019⁵³, Felgner et al, Proc Natl Acad Sci. 1987⁵⁴, Rahimi et al, Bratisl Lek Listy. 2018⁵⁵, Pardi et al. J Control Release. 2015⁵⁶.

In another embodiment, the transfection reagent is a cationic polymers or cationic lipid reagent. In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin® or Lipofectamine®. In another embodiment, the transfection reagent is in vivo-jetRNA® or in vivo-jetPEI®. In another embodiment, the transfection reagent forms a liposome. Liposomes can increase intracellular stability, increase uptake efficiency and improve biological activity. In another embodiment, the transfection reagent forms solid-lipid nanoparticles (SLNs or LNPs), polymers-based nanoparticles such polyethylenimine (PEI)-based nanoparticles; lipopeptides-based nanoparticles such as lipid 1,2-dilinoleyloxy-3-dimethylaminopropane (DLin-DMA)-based nanoparticles, dilinoleylmethyl-4-dimethylaminobutyrate (DLin-MC3-DMA)-based nanoparticles, ALC-0315-based nanoparticles, ALC-0159-based nanoparticles SM-102-based nanonparticles. In another embodiment, the transfection reagent is any other transfection reagent known in the art. Transfection reagent according to the invention includes also transfection reagents as described in patent WO2017049245, WO2018081480 and WO2021016430.

In particular embodiment, the nucleic acid encoding a protein of interest is comprised in a vector.

Thus, in particular embodiment, the present invention relates also to a method for modulating tear film composition in a subject in need thereof, comprising administering into the lacrimal gland of said subject a vector comprising a nucleic acid encoding a protein of interest.

In particular embodiment, the present invention relates to a method for modulating tear film composition in a subject in need thereof, comprising administering into the lacrimal gland of said subject a vector comprising DNA or RNA encoding a protein of interest, and more particular a messenger ribonucleic acid (mRNA) encoding a protein of interest.

As used herein, the term “vector” has its general meaning in the art and refers to the vehicle or agents by which a nucleic acid molecule can be introduced into cells, so as to transform/transfect the cell and promote expression (e.g. transcription and/or translation) of the introduced sequence.

According to the invention, vectors include viral vectors or non-viral vectors.

In some embodiments, the method according to the invention, wherein the vector is a viral or non-viral vector.

Non-viral vectors mainly comprise chemical systems that are not of viral origin and generally include chemical methods such as cationic liposomes and polymers. Non viral vectors useful in the practice of the present invention has very well known in the art. According to the invention non-viral vector includes transfection agents as defined above.

According to the invention, non viral vectors include but are not limited to liposomes such as cationic liposomes, solid-lipid nanoparticles (SLNs or LNPs) such as [(4-hydroxybutyl) azanediyl] di(hexane-6,1-diyl) bis(2-hexyldecanoate)-based nanoparticles; niosomes; polymers such as cationic polymers; polymers-based nanoparticles such polyethylenimine (PEI)-based nanoparticles; lipopeptides-based nanoparticles such as lipid 1,2-dilinoleyloxy-3-dimethylaminopropane (DLin-DMA)-based nanoparticles, dilinoleylmethyl-4-dimethylaminobutyrate (DLin-MC3-DMA)-based nanoparticles, ALC-0315-based nanoparticles, ALC-0159-based nanoparticles SM-102-based nanonparticles and; and chitosans as described in Toualbi L, et al. International Journal of Molecular Sciences, Maier.M et al. Molecular Therapy (2013), Shriane D et al. Biol Pharm Bull (2018). Non viral vectors according to the invention include also the non-viral vectors described in patent WO2017049245, WO2018081480 and WO2021016430.

In some embodiments, the method according to the invention, wherein the non-viral vector is cationic a polymers-based nanoparticle, and more particularly is a polyethylenimine (PEI)-based nanoparticle.

In some embodiments, the method according to the invention, wherein the non-viral vector is in vivo-jetRNA® or in vivo-jetPEI®.

In some embodiments, the method according to the invention, wherein the vector is a non-viral vector comprising ribonucleic acid (RNA) or deoxyribonucleic acid (DNA) encoding a protein of interest and more particularly mRNA encoding a protein of interest. In some embodiments, the method according to the invention, wherein the vector is a non-viract vector when the acid nucleic encodes for NGF or EGF.

In some embodiments, the method according to the invention, wherein the vector is a non-viral vector comprising messenger ribonucleic acid (mRNA) encoding EGF or NGF.

Thus, in a particular embodiment, the present invention relates to a method for modulating tear film composition in a subject in need thereof, comprising administering into the lacrimal gland of said subject a non-viral vector comprising a messenger ribonucleic acid (mRNA) encoding a protein of interest, and in particular mRNA encoding EGF or NGF.

In some embodiments, the method according to the invention, the vector is a viral vector. Viral vectors useful in the practice of the present invention can be constructed utilizing methodologies well known in the art of molecular biology. Typically, viral vectors carrying nucleic acide encoding the protein of interest, suitable regulatory elements and elements necessary for production of viral proteins which mediate cell transduction. Examples of viral vector include but are not limited to retrovirus, adenovirus, adeno-associated virus (AAV), herpes virus, pox virus, human foamy virus (HFV), and lentivirus.

In some embodiments, the method according to the invention, wherein the viral vector is a lentivirus (LV) vector, adenovirus vector or an adeno-associated virus (AAV) vector.

As used herein, the term “lentivirus” refers to enveloped RNA particles measuring approximately 120 nm in size are efficient drug delivery tools and more particularly gene delivery tools. The LV binds to, and enters into target cells through its envelope proteins which confer its pseudotype. Once the LV has entered into the cells, it releases its capsid components and undergoes reverse transcription of the lentiviral RNA before integrating the proviral DNA into the genome of target cells. Non-integrative lentiviral vectors have been generated by modifying the properties of the vector integration machinery and can be used for transient gene expression. Virus-like particles lacking a provirus have also been generated and can be used to deliver proteins or messenger RNA. LV can be used for example, for gene addition, RNA interference, exon skipping or gene editing. All of these approaches can be facilitated by tissue or cell targeting of the LV via its pseudotype.

Lentivirus-like particles are described for example in (Aoki et al., 2011; Kaczmarczyk et al., 2011; McBurney et al., 2006; Muratori et al., 2010). Examples of lentivirus-like particles are VLPs generated by co-expressing in producer cells, a syncytin protein with a gag fusion protein (Gag fused with the gene of interest). The drug and/or syncytin may be, either displayed on the surface of the particles, or enclosed (packaged) into the particles. The syncytin protein is advantageously displayed on the surface of the particles, such as coupled to the particles or incorporated into the envelope of (enveloped) virus particles or virus-like particles to form pseudotyped enveloped virus particles or virus-like particles. The drug is coupled to the particles or packaged into the particles. For example, the drug is coupled to viral capsids or packaged into viral capsids, wherein said viral capsids may further comprise an envelope, preferably pseudotyped with syncytin. In some preferred embodiments, the drug is packaged into the particles pseudotyped with syncytin protein. The drug which is packaged into particles is advantageously a heterologous gene of interest which is packaged into viral vector particles, preferably retroviral vector particles, more preferably lentiviral vector particles.

As used herein, the term “adenovirus” refers to medium-sized (90-100 nm), nonenveloped (without an outer lipid bilayer) viruses with an icosahedral nucleocapsid containing a double stranded DNA genome.

In a particular embodiment, the method according to the invention, wherein the viral vector is an adeno-associated virus (AAV) vector.

As used herein, the term “AAV vector” refers to a vector derived from an adeno-associated virus serotype, including without limitation AAV1, AAV2, AAV3, AAV4, AA5, AAV6, AAV7, AAV8, AAV9, AAVrh10 or any other serotypes of AAV that can infect humans, monkeys or other species. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the nucleic acid molecule of the present invention and a transcriptional termination region. The control elements are selected to be functional in a mammalian cell. The resulting construct which contains the operatively linked components is bounded (5′ and 3′) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8 or AAV9. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In some embodiments, the AAV vector of the present invention is a double-stranded, self-complementary AAV (scAAV) vector. Alternatively to the use of single-stranded AAV vector, self-complementary vectors can be used. The efficiency of AAV vector in terms of the number of genome-containing particles required for transduction, is hindered by the need to convert the single-stranded DNA (ssDNA) genome into double-stranded DNA (dsDNA) prior to expression. This step can be circumvented through the use of self-complementary vectors, which package an inverted repeat genome that can fold into dsDNA without the requirement for DNA synthesis or base-pairing between multiple vector genomes. Resulting self-complementary AAV (scAAV) vectors have increased resulting expression of the transgene. For an overview of AAV biology, ITR function, and scAAV constructs, see McCarty D M. Self-complementary AAV vectors; advances and applications. Mol. Ther. 2008 October; 16 (10): at pages 1648-51, first full paragraph, incorporated herein by reference for disclosure of AAV and scAAV constructs, ITR function, and role of ATRS ITR in scAAV constructs. A rAAV vector comprising a ATRS ITR cannot correctly be nicked during the replication cycle and, accordingly, produces a self-complementary, double-stranded AAV (scAAV) genome, which can efficiently be packaged into infectious AAV particles. Various rAAV, ssAAV, and scAAV vectors, as well as the advantages and drawbacks of each class of vector for specific applications and methods of using such vectors in gene transfer applications are well known to those of skill in the art (see, for example, Choi V W, Samulski R J, McCarty D M. Effects of adeno-associated virus DNA hairpin structure on recombination. J. Virol. 2005 June; 79 (11): 6801-7; McCarty D M, Young S M Jr, Samulski R J. Integration of adeno-associated virus (AAV) and recombinant AAV vectors. Annu Rev Genet. 2004; 38:819-45; McCarty D M, Monahan P E, Samulski R J. Self-complementary recombinant adeno-associated virus (scAAV) vectors promote efficient transduction independently of DNA synthesis. Gene Ther. 2001 August; 8 (16): 1248-54; and McCarty D M. Self-complementary AAV vectors; advances and applications. Mol. Ther. 2008 October; 16 (10): 1648-56; all references cited in this application are incorporated herein by reference for disclosure of AAV, rAAV, and scAAV vectors).

In some embodiment, the AAV vector is an AAV2/9 vector or an AAV2/5 vector. AAV2/9 vector denotes that the AAV2 genome carrying the transgene (i.e nucleic acid encoding for the protein of interest) was packaged in an AAV9 capsid. An AAV2/5 vector refers to a AAV2 genome carrying the transgene (i.e e nucleic acid encoding for the protein of interest) packaged in an AAV5 capsid. Scalable production of such rAAV are well known to those of skill in the art (Blessing D, et al. Scalable production of AAV vectors in orbitally shaken HEK293 cells. Mol Ther Methods Clin Dev. 2019 Jun. 14; 13:14-26.

Thus, in some embodiments, the present invention relates to a method for modulating tear film composition in a subject in need thereof, comprising administering into the lacrimal gland of said subject a viral vector comprising nucleic acid encoding for a protein of interest.

In particular embodiment, the present invention relates to a method for modulating tear film composition in a subject in need thereof, comprising administering into the lacrimal gland of said subject an AAV vector comprising a deoxyribonucleic acid (DNA) encoding for a protein of interest.

In particular embodiment, concerning all the methods of the invention, the AAV vector comprising deoxyribonucleic acid (DNA) or ribonucleic acid (RNA) encoding for a protein of interest is administered alone. In particular embodiment, concerning all the methods of the invention, the AAV vector comprising deoxyribonucleic acid (DNA) or ribonucleic acid (RNA) encoding for a protein of interest is not administered in combination with nicotinic acetylcholine receptor (nAChR) agonist.

In particular embodiment, the present invention relates to a method for modulating tear film composition in a subject in need thereof, comprising administering into the lacrimal gland of said subject an AAV2/9 vector comprising a deoxyribonucleic acid (DNA) encoding for a protein of interest.

In particular embodiment, the present invention relates to a method for modulating tear film composition in a subject in need thereof, comprising administering into the lacrimal gland of said subject an AAV2/9 vector comprising a deoxyribonucleic acid (DNA) encoding for a NGF.

In particular embodiment, concerning all the methods of the invention, the AAV2/9 vector comprising deoxyribonucleic acid (DNA) encoding for NGF is administered alone. In particular embodiment, concerning all the methods of the invention, the AAV2/9 vector comprising deoxyribonucleic acid (DNA) encoding for NGF is not administered in combination with nicotinic acetylcholine receptor (nAChR) agonist.

The inventors shows that a single AAV2/9 CAG-mNGF injection in the lacrimal gland stimulate the reinnervation of the cornea, following an epithelial abrasion.

Thus the methods of the invention are suitable for improving corneal reinnervation.

The methods of the invention are also suitable for improving corneal wound healing.

The methods of the invention are also suitable for increasing tear production.

Thus, in another aspect the present invention relates to a method for improving corneal reinnervation in a subject in need thereof, comprising administering into the lacrimal gland of said subject a nucleic acid encoding a protein of interest.

The inventors shows also that a single AAV2/9 CAG-mNGF injection in the lacrimal gland increase the tear volume.

Thus, in another aspect the present invention relates to a method for increasing tear production in a subject in need thereof, comprising administering into the lacrimal gland of said subject a nucleic acid encoding a protein of interest.

Thus, in another aspect the present invention relates to a method for improving corneal wound healing in a subject in need thereof, comprising administering into the lacrimal gland of said subject a nucleic acid encoding a protein of interest.

In some embodiment, regarding the method for improving corneal reinnervation and/or corneal wound healing and/or increasing tear production, the nucleic acid is deoxyribonucleic acid (DNA) or ribonucleic acid (RNA), and more particularly messenger ribonucleic acid (mRNA).

In some embodiment, regarding the method for improving corneal reinnervation and/or corneal wound healing and/or increasing tear production, the protein of interest is selected in the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), bone morphogenetic protein 6 (BMP6), insulin-like growth factor 1 (IGF-1), insulin-like growth factor 2 (IGF-2), glial-cell derived neurotrophic factor (GDNF) and transforming growth factor (TGF).

In some embodiment, regarding the method for improving corneal reinnervation and/or corneal wound healing and/or increasing tear production, the protein of interest is nerve growth factor (NGF).

In some embodiments, regarding the method for improving corneal reinnervation and/or corneal wound healing and/or increasing tear production, the messenger ribonucleic acid (mRNA) encoding for a protein of interest such as NGF is administered gland simultaneously, separately or sequentially with a transfection reagent, such as in vivo-RNAjet.

In some embodiments, regarding the method for improving corneal reinnervation and/or corneal wound healing and/or increasing tear production, the messenger ribonucleic acid (mRNA) encoding for a protein of interest such as NGF is further mixed with a transfection reagent prior to the administration into lacrimal gland.

Thus, in particular embodiment, the invention refers to a method for improving corneal reinnervation and/or corneal wound healing and/or increasing tear production in a subject in need thereof, comprising administering into the lacrimal gland of said subject a messenger ribonucleic acid (mRNA) encoding for a protein of interest such as NGF, wherein said mRNA is administered simultaneously, separately or sequentially with a transfection reagent.

In particular embodiment, the invention refers to a method for improving corneal reinnervation and/or corneal wound healing and/or increasing tear production in a subject in need thereof, comprising administering into the lacrimal gland of said subject a messenger ribonucleic acid (mRNA) encoding a protein of interest, wherein said mRNA is administered simultaneously, separately or sequentially with a transfection reagent, and wherein the protein of interest is selected in the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), bone morphogenetic protein 6 (BMP6), insulin-like growth factor 2 (IGF-2), glial-cell derived neurotrophic factor (GDNF) and transforming growth factor (TGF).

In particular embodiment, the invention refers to a method for improving corneal reinnervation and/or corneal wound healing and/or increasing tear production in a subject in need thereof, comprising administering into the lacrimal gland of said subject a messenger ribonucleic acid (mRNA) encoding a protein of interest, wherein said mRNA is further mixed with a transfection reagent prior to the administration into lacrimal gland, and wherein the protein of interest is selected in the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), bone morphogenetic protein 6 (BMP6), insulin-like growth factor 2 (IGF-2), glial-cell derived neurotrophic factor (GDNF) and transforming growth factor (TGF).

In particular embodiment, the invention refers to a method for corneal reinnervation and/or corneal wound healing and/or increasing tear production in a subject in need thereof, comprising administering into the lacrimal gland of said subject a messenger ribonucleic acid (mRNA) encoding NGF, wherein said mRNA is administered simultaneously, separately or sequentially with a transfection reagent.

In some embodiments, regarding the method for improving corneal reinnervation and/or corneal wound healing and/or increasing tear production, the nucleic acid is comprised in a vector.

In some embodiments, regarding the method for improving corneal reinnervation and/or corneal wound healing and/or increasing tear production, the vector is a viral or non-viral vector.

In some embodiments, regarding the method for improving corneal reinnervation and/or corneal wound healing and/or increasing tear production the vector is a non-viral vector comprising deoxyribonucleic acid (DNA) or ribonucleic acid (RNA) encoding NGF and more particularly messenger RNA encoding a protein of interest.

In some embodiments, regarding the method for improving corneal reinnervation and/or corneal wound healing and/or increasing tear production, the viral vector is a an adeno-associated virus (AAV) vector and more particularly AAV2/9.

In some embodiments, regarding the method for improving corneal reinnervation and/or corneal wound healing and/or increasing tear production, wherein the viral vector is an AAV2/9 vector comprising deoxyribonucleic acid (DNA) or ribonucleic acid (RNA) encoding a protein of interest.

Thus, in particular embodiment, the invention refers to a method for improving corneal reinnervation and/or corneal wound healing and/or increasing tear production in a subject in need thereof, comprising administering into the lacrimal gland of said subject an AAV2/9 vector comprising deoxyribonucleic acid (DNA) or ribonucleic acid (RNA) encoding a protein of interest.

Thus, in particular embodiment, the invention refers to a method for corneal reinnervation and/or corneal wound healing and/or increasing tear production in a subject in need thereof, comprising administering into the lacrimal gland of said subject an AAV2/9 vector comprising deoxyribonucleic acid (DNA) or ribonucleic acid (RNA) encoding a protein of interest, wherein the protein of interest is selected in the group consisting of nerve growth factor (NGF), neuropilin 2 (NRP2), semaphorin 3C SEMA3C, pigment epithelium-derived factor (PEDF), glial-cell derived neurotrophic factor (GDNF) and thyroglobuline.

Thus, in particular embodiment, the invention refers to a method for improving corneal reinnervation and/or corneal wound healing and/or increasing tear production in a subject in need thereof, comprising administering into the lacrimal gland of said subject an AAV2/9 vector comprising deoxyribonucleic acid (DNA) or ribonucleic acid (RNA) encoding NGF.

In particular embodiment, the an AAV2/9 vector comprising deoxyribonucleic acid (DNA) or ribonucleic acid (RNA) encoding aprotein of interest is not administered in combination with nicotinic acetylcholine receptor (nAChR) agonist. In particular embodiment, the an AAV2/9 vector comprising deoxyribonucleic acid (DNA) or ribonucleic acid (RNA) encoding a protein of interest is administered alone.

In particular embodiment, the an AAV2/9 vector comprising deoxyribonucleic acid (DNA) or ribonucleic acid (RNA) encoding a NGF is not administered in combination with nicotinic acetylcholine receptor (nAChR) agonist. In particular embodiment, the an AAV2/9 vector comprising deoxyribonucleic acid (DNA) or ribonucleic acid (RNA) encoding a NGF is administered alone.

The methods of the invention are also particularly suitable to treat corneal defects in subject in need thereof.

Thus, the present invention relates to a method for treating corneal defects in subject in need thereof comprising administering into the lacrimal gland to said subject an effective amount of a nucleic acid encoding a protein of interest.

In other word, the invention relates to a nucleic acid encoding a protein of interest for use for treating corneal defects in subject in need thereof, wherein said nucleic acid is administered into the lacrimal gland.

In some embodiment, the nucleic acid is deoxyribonucleic acid (DNA) or ribonucleic acid (RNA), and more particularly messenger RNA.

In some embodiments, the nucleic acid is administered into the lacrimal gland simultaneously, separately or sequentially with a transfection reagent.

In some embodiments, the messenger RNA acid is administered into the lacrimal gland simultaneously, separately or sequentially with a transfection reagent.

In some embodiments, the messenger RNA acid is further mixed with a transfection reagent prior to the administration into lacrimal gland.

In some embodiments, the nucleic acid is comprised in a vector.

In some embodiments, the vector is a viral or non-viral vector.

In some embodiments, the viral vector is an adeno-associated virus (AAV) vector comprising deoxyribonucleic acid (DNA) encoding a protein of interest.

In some embodiments, the AAV vector is an AAV2/9 or AAV2/5 vector

Thus, in particular embodiments, the inventions relates to an AAV2/9 vector comprising deoxyribonucleic acid (DNA) encoding a protein of interest for use for treating corneal defects in subject in need thereof.

Thus, in particular embodiments, the inventions relates to an AAV2/9 vector comprising deoxyribonucleic acid (DNA) encoding a protein of interest for use for treating corneal defects in subject in need thereof, wherein the AAV2/9 vector is not administered with nicotinic acetylcholine receptor (nAChR) agonist.

In another embodiments, the vector is a non-viral vector comprising messenger ribonucleic acid (RNA) encoding a protein of interest.

Thus, in particular embodiment, the inventions relates to a non-viral vector comprising messenger ribonucleic acid (mRNA) encoding a protein of interest for use for treating corneal defects in subject in need thereof.

In particular embodiment, the inventions relates to a messenger ribonucleic acid (mRNA) encoding a protein of interest for use for treating corneal defects in subject in need thereof, wherein the mRNA encoding a protein of interest is administered into the lacrimal gland simultaneously, separately or sequentially with a transfection reagent.

In some embodiment, the protein of interest is selected from the group consisting in nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), bone morphogenetic protein 6 (BMP6), insulin-like growth factor 1 (IGF-1), insulin-like growth factor 2 (IGF-2), glial-cell derived neurotrophic factor (GDNF) and transforming growth factor (TGF).

In particular embodiment, the protein of interest is selected from the group consisting in nerve growth factor (NGF), vascular Endothelial Growth Factor A (VEGF-A), epidermal growth factor (EGF), neuropilin 2 (NRP2), semaphorin 3C SEMA3C, pigment epithelium-derived factor (PEDF), glial-cell derived neurotrophic factor (GDNF) and thyroglobuline.

In particular embodiment, the protein of interest is NGF and the tear production is increased.

Thus, in particular embodiment, the invention relates to a method for treating corneal defects by increasing tear production in subject in need thereof comprising administering into the lacrimal gland to said subject an effective amount of a nucleic acid encoding a protein of interest.

Thus, in particular embodiment, the invention relates to a method for treating corneal defects by increasing tear production in subject in need thereof comprising administering into the lacrimal gland to said subject an effective amount of a nucleic acid encoding NGF

In particular embodiment, the invention relates to a method for treating corneal defects by improving corneal reinnervation and/or corneal wound healing in subject in need thereof comprising administering into the lacrimal gland to said subject an effective amount of a nucleic acid encoding a protein of interest.

Thus, in particular embodiment, the invention relates to a method for treating corneal defects by improving corneal reinnervation and/or corneal wound healing in subject in need thereof comprising administering into the lacrimal gland to said subject an effective amount of a nucleic acid encoding a protein of interest, wherein the protein of interest is selected from the group consisting in nerve growth factor (NGF), vascular Endothelial Growth Factor A (VEGF-A), epidermal growth factor (EGF), neuropilin 2 (NRP2), semaphorin 3C SEMA3C, pigment epithelium-derived factor (PEDF), thyroid hormone, and glial-cell derived neurotrophic factor (GDNF).

As used herein, the term “corneal defects”, has its general meaning in the art and refers to a broad range of diseases, including physical wounds corneal (e.g abrasion), dry eye diseases, diabetes such as diabetic keratopathy, fuchs' endothelial corneal dystrophy, bullous keratopathy, corneal thinning disorders, ICE syndrome or neurotrophic keratitis (NK).

In some embodiment, the corneal defects is physical wounds corneal, dry eye diseases, diabetic keratopathy, corneal thinning disorders, ICE syndrome or neurotrophic keratitis (NK).

In particular embodiment, the corneal defects is drye eye diseases or physical wouds corneal.

Thus, the methods of the invention are particularly suitable for treating drye eye diseases by increasing tear production.

Thus, in particular embodiment, the invention relates to a method for treating drye eye diseases by increasing tear production in subject in need thereof comprising administering into the lacrimal gland to said subject an effective amount of a nucleic acid encoding a protein of interest.

Thus, in particular embodiment, the invention relates to a method for treating drye eye diseases by increasing tear production in subject in need thereof comprising administering into the lacrimal gland to said subject an effective amount of an AVV2/9 comprising DNA encoding a protein of interest, and more particularly DNA encoding NGF.

Thus, in particular embodiment, the invention relates to a method for treating drye eye diseases by increasing tear production in subject in need thereof comprising administering into the lacrimal gland to said subject an effective amount of a mRNA a protein of interest, and more particularly DNA encoding NGF.

As used herein, the term “dry eye disease” or “dry eye symptom”, has its general meaning in the art and refers to a generic ocular disorder. Dry eye disease (DED) has become common on a global scale in recent years. There is a wide prevalence of DED in different countries based on various ethnicities and environment. In addition to advanced age and gender, such factors as living at high altitude, smoking, pterygium, prolonged use of consumer electronics or overingesting of caffeine or multivitamins are considered to be the major risk factors of DED (Kuo et al, 2019). Dry eye symptom can be idiopathic (without apparent cause) Furthermore, a various broad of pathology can caused dry eye symptom. Dry eye symptom can be caused by alacrima, xerophthalmia, lacrimal gland ablation, sensory denervation, collagen vascular diseases and immune disease such as polychondritis, allergic eye diseas, sarcoidosis, thyroid disorders, rheumatoid arthritis, graft vs. host disease, diabetes, neurotrophic keratitis and systemic lupus erythematosus. Sjögren syndrome are mainly associated with drye eye disease. Drugs such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine can cause or worsen dry eye symptom. Drye eye symptom can also result from nocturnal lagophthalmos or insufficient rate of blinking. Abnormalities of the mucin tear layer caused by vitamin A deficiency, trachoma, diphtheric keratoconjunctivitis, mucocutaneous disorders and certain topical medications are also causes of dry eye symptom. Dry eye symptom can also result after eye surgery, such as refractive surgeries or cornea transplant.

In some embodiment, dry eye symptom is caused by the Sjögren syndrome, diabetic keratopathy, eye surgery or neurotrophic keratitis.

As used herein, the term “Sjögren syndrome”, has its general meaning in the art and refers to an autoimmune disease or disorder in which immune cells attack the glands that produce tears and saliva. The hallmark symptoms of the disorder are dry mouth and dry eyes. In addition, Sjögren's syndrome may cause skin, nose, and vaginal dryness, and may affect other organs of the body including the kidneys, blood vessels, lungs, liver, pancreas, and brain. Sjögren's syndrome can exist as a primary disorder (“primary Sjögren's syndrome”) or as a secondary disorder (“secondary Sjögren's syndrome”) that is associated with and/or secondary to other autoimmune disorders including rheumatic disorders such as rheumatoid arthritis, systemic lupus, polymyositis, scleroderma, and autoimmune hepatitis, lymphomas such as non-Hodgkin's lymphoma, and endocrine disorders such as thyroiditis. The term “Sjögren's syndrome” as used herein applies to Sjögren's syndrome no matter what the stage, including both primary and secondary Sjögren's syndrome.

As used herein, the term “neurotrophic keratitis” (NK), has its general meaning in the art and refers to a degenerative disease characterized by corneal sensitivity reduction, spontaneous epithelium breakdown, and impairment of corneal healing. Several causes of NK includes herpetic keratitis, diabetes, and ophthalmic and neurosurgical procedures. NK is characterized by decreased or absent corneal sensation, leading to epithelial breakdown, impairment of healing, and ultimately corneal ulceration, melting and perforation.

As used herein, the term “diabetic keratopathy” has its general meaning in the art and refers to the main ocular complications from diabetes mellitus. Diabetic keratopathy exhibits several clinical manifestations, including persistent corneal epithelial erosion, superficial punctate keratopathy, delayed epithelial regeneration, and decreased corneal sensitivity, that may lead to compromised visual acuity or permanent vision loss.

As used herein, the term “keratoconus” has its general meaning in the art and refers to thinning of cornea which gradually bulges outward into a cone shape. A cone-shaped cornea causes blurred vision and may cause sensitivity to light and glare. Keratoconus usually affects both eyes, though it often affects one eye more than the other.

As used herein, the term “ICE syndrome” for “Iridocorneal Endothelial (ICE) Syndrome” has its general meaning in the art and refers to a group of ophthalmic disorder that involves an irregular corneal endothelium that can lead to varying degrees of corneal edema, iris atrophy, and secondary angle-closure glaucoma.

The methods of the invention is also particurlary suitable to prevent or treat corneal defect caused by eye surgery, such as refractive surgery (PRK or LASIK) or cornea transplant.

As used herein, the term “eye surgery” include various corneal surgical procedures. According to the invention, eye surgery includes corneal transplantation, amniotic membrane transplantation, limbal stem cell transplantation, phototherapeutic kerarectomy (PTK), photorefractive keratectomy (PRK), Laser in Situ Keratomileusis (LASIK) eye surgery and removal of conjunctival lesions.

The methods of the invention are particularly suitable for treating physical wounds corneal by improving corenal reinnervation and/or corneal wounds healing.

Thus, in particular embodiment, the invention relates to a method for treating physical wounds corneal by improving corenal reinnervation and/or corneal wounds healing in subject in need thereof comprising administering into the lacrimal gland to said subject an effective amount of a nucleic acid encoding a protein of interest.

Thus, in particular embodiment, the invention relates to a method for treating physical wounds corneal by improving corenal reinnervation and/or corneal wounds healing in subject in need thereof comprising administering into the lacrimal gland to said subject an effective amount of an AVV2/9 comprising DNA encoding a protein of interest, and more particularly DNA encoding NGF.

Thus, in particular embodiment, the invention relates to a method for treating physical wounds corneal by improving corenal reinnervation and/or corneal wounds healing in subject in need thereof comprising administering into the lacrimal gland to said subject an effective amount of a mRNA a protein of interest, and more particularly DNA encoding NGF

As used herein, the term “therapeutically effective amount” refers to an amount sufficient to produce the desired physiological effect or amount capable of achieving the desired result, particularly for treating the disorder or disease condition, including reducing or eliminating one or more symptoms or manifestations of the disorder or disease. The effective amount will vary with the age, general condition of the subject, the severity of the condition being treated, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. According to the invention, the method of the invention allows to ameliorate/improve corneal reinnervation.

The treatment may be administered to a subject having a medical disorder or who ultimately may acquire the disorder, in order to prevent, cure, delay the onset of, reduce the severity of, or ameliorate one or more symptoms (i.e dry eye) of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiment, the vector of the invention is administered by a local injection into lachrymal gland.

### Pharmaceutical Composition

The nucleic acid encoding a protein of interest according to the invention may be administrated in the form of a pharmaceutical compositions comprising pharmaceutically acceptable excipients, and optionally sustained-release matrices, such as biodegradable polymers

Accordingly, the invention relates to a pharmaceutical composition comprising a nucleic acid encoding a protein of interest for use for modulating tear film composition in a subject in need thereof.

The invention also relates to a pharmaceutical composition comprising a nucleic acid encoding a protein of interest for use for improving corneal reinnervation and/or increasing tear production in a subject in need thereof.

The invention also relates to a pharmaceutical composition comprising a nucleic acid encoding a protein of interest for use for treating corneal defects in a subject in need thereof.

In particular embodiment, the nucleic acid (RNA) is messenger ribonucleic acid, and more particularly a messenger ribonucleic acid encoding for NGF or EGF.

In particular embodiment, the pharmaceutical composition further comprises a transfection reagents

In particular embodiment, the nucleic acid (RNA) is administered with transfection reagent.

Thus, in particular embodiment, the pharmaceutical composition comprises a messenger ribonucleic acid, and more particularly a messenger ribonucleic acid encoding for NGF or EGF, wherein the pharmaceutical composition is administered with transfection reagent.

The vector comprising a nucleic acid encoding a protein of interest according to the invention may be administrated in the form of a pharmaceutical compositions comprising pharmaceutically acceptable excipients, and optionally sustained-release matrices, such as biodegradable polymers.

Accordingly, the invention relates to a pharmaceutical composition comprising a vector comprising a nucleic acid encoding a protein of interest for use for modulating tear film composition in a subject in need thereof.

The invention also relates to a pharmaceutical composition comprising a vector comprising a nucleic acid encoding a protein of interest for use for improving corneal reinnervation and/or corneal wound healing and/or increasing tear production in a subject in need thereof.

The invention also relates to a pharmaceutical composition comprising a vector comprising a nucleic acid encoding a protein of interest for use for treating corneal defects in a subject in need thereof.

In some embodiments, the vector is a viral or non-viral vector.

In some embodiment, the nucleic acid is deoxyribonucleic acid (DNA) or ribonucleic acid (RNA).

In some embodiment, the ribonucleic acid (RNA) is messenger ribonucleic acid.

In some embodiments, the viral vector is an adeno-associated virus (AAV) vector.

In some embodiments, the AAV vector is an AAV2/9 or AAV2/5 vector.

In some embodiments, the AAV vector comprises deoxyribonucleic acid (DNA) encoding a protein of interest.

Thus, in particular embodiments, the inventions relates to an AAV2/9 vector comprising deoxyribonucleic acid (DNA) encoding a protein of interest for use for treating corneal defects in subject in need thereof.

In another embodiments, the non-viral vector comprises messenger ribonucleic acid (RNA) encoding a protein of interest.

In some embodiment, the protein of interest is selected in the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), bone morphogenetic protein 6 (BMP6), insulin-like growth factor 1 (IGF-1), insulin-like growth factor 2 (IGF-2), glial-cell derived neurotrophic factor (GDNF) and transforming growth factor (TGF).

In some embodiment, the protein of interest is NGF.

In some embodiment, the pharmaceutical composition does not compises nicotinic acetylcholine receptor (nAChR) agonist.

In some embodiment, the pharmaceutical composition for use according to the invention is not administered in combination with nicotinic acetylcholine receptor (nAChR) agonist.

In a particular embodiment, according to all the aspects of the invention, the pharmaceutical composition is suitable for a local administration to the subject to be treated, such as is suitable for an administration into the lacrimal gland of the subject to be treated.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically” or “pharmaceutically acceptable” refers to medium and compositions that do not produce an adverse, allergic or other untoward reaction when administered to a mammal, especially a human, as appropriate. A pharmaceutically acceptable medium comprises any of standard pharmaceutically accepted mediums known to those of ordinary skill in the art, in particular in formulating pharmaceutical compositions to be administered to the eye.

In some embodiment, the pharmaceutical composition of the invention is administered by a local injection. In some embodiment, the pharmaceutical composition of the invention is injected into the gland lacrimal

In some embodiment, the pharmaceutical composition is injected by needle linked to a syringe, and more particulary by a beveled needle linked to a syringe, and more particularly by a 34-gauge beveled needle linked to a syringe.

The invention will be further illustrated by the following figures and examples. However, these examples and figures should not be interpreted in any way as limiting the scope of the present invention.

### FIGURES

FIG. 1. GFP is detected in AAV2/9 and AAV2/5 CAG-GFP-injected LG and dosed by Western blot. Quantification of GFP/total protein ratio in LG lysates (n=3 mice per group). Results are expressed as the mean±SD. Statistical analysis using Brown-Forsythe and Welch ANOVA tests followed by Dunnett's T3 multiple comparisons test. ** p=0.0026 between AAV2/9 empty capsid and AAV2/9 CAG-GFP groups, ** p=0.0077 between AAV2/5 empty capsid and AAV2/5 CAG-GFP groups, ** p=0.0018 between AAV2/9 CAG-GFP and AAV2/5 CAG-GFP groups; ns, not significant; A. U., arbitrary units.

FIG. 2. Injections of AAV2/5 or AAV2/9 CAG-mNGF in the murine LG significantly increase the level of mNGF in tears. mNGF protein levels were analyzed by ELISA in the tears of AAV2/9 or AAV2/5 CAG-mNGF-injected mice in the right LG at 12 weeks old, when compared to AAV2/9 or AAV2/5 empty capsid-injected mice, 1 month post injection respectively (10¹⁰ vg/LG in 3 μl, n=5 mice per group). A. Quantification of the mNGF level in the tears of injected mice. Results are expressed as the mean±SD. Statistical analysis using Brown-Forsythe and Welch ANOVA tests followed by Dunnett's T3 multiple comparisons test. ** p=0.0065 between AAV2/9 empty capsid and AAV2/9 CAG-mNGF groups, * p=0.0464 between AAV2/5 empty capsid and AAV2/5 CAG-mNGF groups, ** p=0.0085 between AAV2/9 CAG-mNGF and AAV2/5 CAG-mNGF groups.

FIG. 3. Dose response and kinetic studies reveal the correlation between mNGF protein level in the tears and the AAV2/9-injected dose, and the detection of mNGF in tears for at least 6 months. For the dose response study, mNGF protein levels were analyzed by ELISA in the tears of AAV2/9 CAG-mNGF-injected mice in the right LG at 12 weeks old by increasing dose of vector (10⁹, 10¹⁰, and 10¹¹ vg/LG in 3 μl, n=5 mice per dose), when compared to the tears of AAV2/9 empty capsid-injected mice 30 days post injection. For the long-term kinetic study, mNGF protein levels were analyzed by ELISA in the tears of AAV2/9 CAG-mNGF-injected mice in the right LG at 12 weeks old (10¹¹ vg/LG in 3 μl), when compared to the tears of AAV2/9 empty capsid-injected mice before injection and 7, 15, 30, 60, 90, 120 and 150 days post injection (n=3 or 5 for AAV2/9 empty capsid-injected mice and AAV2/9 CAG-mNGF-injected mice respectively). A. Quantification by ELISA of the mNGF protein level in the tears 30 days post injection following the indicated increasing dose of injected vector. Results are expressed as the mean±SD. Statistical analysis using Brown-Forsythe and Welch ANOVA tests followed by Dunnett's T3 multiple comparisons test. * p=0.0107 and * p=0.0341 between the doses 10⁹ and 10¹⁰ vg/LG and between the doses 10¹⁰ and 10¹¹ vg/LG respectively. ns, not significant. B. Quantification by ELISA of the mNGF protein level in the tears up to 6 months post injection at the indicated days post injection. Statistical analysis using repeated measures two-way ANOVA test followed by Sidak's multiple comparisons test. ** p=0.0012, p=0.0574, * p=0.0299, ** p=0.0356, * p=0.0410, * p=0.0346, * p=0.0454, between AAV2/9 empty capsid and AAV2/9 CAG-mNGF groups 7, 15, 30, 60, 90, 120 and 150 days post injection respectively. ns, not significant.

FIG. 4. Biodistribution analysis after the injection of AAV2/9 CAG-mNGF in murine LG reveals a limited diffusion to peripheral organs. AAV2/9 CAG-mNGF was injected in the right LG of 12 week-old mice (10¹¹ vg/LG in 3 μl, n=7 mice). 1 month post injection, tears were collected and mNGF protein level was quantified by ELISA. At the same time, mice were sacrificed, LG, liver and heart were collected and analyzed by qPCR. A. Graph showing the transduction rate expressed in vector genome/diploid genome (vg/dg) above the lowest limit of quantification (LLOQ). Results are expressed as the mean±SD. Statistical analysis using Brown-Forsythe and Welch ANOVA tests followed by Dunnett's T3 multiple comparisons test. **** p<0.0001 between LG and liver, **** p<0.0001 between LG and heart, * p=0.0226 between liver and heart. B. Graph showing the linear correlation for each mouse between the vg/dg in LG and the mNGF protein level (ng/ml) from the statistical linear regression (red line). R2, R squared.

FIG. 5. Injection of AAV2/9 CAG-mNGF in murine LG does not alter tear protein concentration nor corneal integrity. AAV2/9 empty capsid or AAV2/9 CAG-mNGF were injected into murine LG (10¹¹ vg/LG in 3 μl, n=3 and 5 mice respectively). The volume of tears (A), the protein concentration in tears (B) were assessed 1 day before injection and 7, 30, 60, 120 days and 180 post injection. A. Graph showing the volume of tears (μl/min) collected before injection and at the indicated days post injection. Results are expressed as the mean±SD. Statistical analysis using repeated measures two-way ANOVA test followed by Sidak's multiple comparisons test. ** p=0.0036, **** p<0.0001, ** p=0.0057, * p=0.0246 between AAV2/9 empty capsid and AAV2/9 CAG-mNGF groups at 7, 30, 60 and 120 days post injection respectively. ns, not significant. B. Graph showing the protein concentration in tears (mg/ml) collected before injection and at the indicated days post injection. Results are expressed as the mean±SD. Statistical analysis using repeated measures two-way ANOVA test followed by Sidak's multiple comparisons test. ns, not significant.

FIG. 6. Injection of AAV2/9 CAG-mNGF into murine LG does not affect corneal sensitivity. Quantification of the corneal sensitivity (1/g) measured by von Frey test performed both on the contralateral side and the injected side of AAV2/9 empty capsid (white plots, n=7) or AAV2/9 CAG-mNGF (red plots, n=5)-injected mice. Von Frey analysis was performed two weeks post injection. Results are expressed as the mean±SD. Statistical analysis using two-way ANOVA test followed by Sidak's multiple comparisons test. ns, not significant.

FIG. 7. Injection mRNA into murine lacrimal gland. 0.85 μg of mRNA are diluted in the transfection medium (In vivo-Jet for example) and then are incubated for 15 min at room temperature. As complexes are stable 1 hr at room temperature, injections are performed swiftly (0.85 μg of mRNA in 2 μL are injected in each murine LG).

### EXAMPLE 1

### Material & Methods

### Study Design

The goal of this study was to assess the transduction pattern of AAV vector serotypes 2/5 and 2/9 after a single injection into murine LG. Then, to evaluate the efficiency and the safety of a AAV2/9-mediated gene transfer of mNGF in the murine LG, for its secretion in the tear film. The main readouts of this study included: the transduction pattern analyzed by IHC and Western blot, the secretion of mNGF measured in tears by ELISA and Western blot, the AAV2/9 biodistribution by qPCR, the injection biosafety by analyzing the corneal integrity and innervation, the volume of tears and the protein concentration in tears. Experimental groups were sized according to the literature to allow statistical analysis. No outliers were excluded from the study, except mice exhibiting spontaneous eye damages after the surgery or during the experiments. Behavioral data obtained from animals displaying eye damages unrelated to the abrasion procedure during the study were excluded. Scientists who performed the experiments and analysis were blinded to the group's identity. Data were analyzed by those carrying out the experiments and verified by the supervisor.

### Cloning and Vector Production

Cloning of the enhanced GFP (GFP) and the mNGF in pAAV and AAV vector productions were provided by the vector core of the TarGeT (Translational Gene Therapy) Laboratory of Nantes, INSERM UMR 1089 (Nantes University, France). Briefly, single-stranded AAV2/5 and AAV2/9 CAG-GFP, single-stranded AAV2/5 and AAV2/9 CAG-mNGF vectors were obtained from pAAV CAG-GFP and pAAV CAG-mNGF plasmids respectively, containing AAV2 inverted terminal sequences, CAG promoter and BGH poly A signal. AAV2/5 and AAV2/9 CAG-GFP vectors were used to assess the transduction pattern after injection in the murine LG. AAV2/5 and AAV2/9 CAG-mNGF vectors were used to evaluate the efficiency of an AAV-based gene transfer in the LG allowing mNGF secretion in the tear fluid. Single-stranded vectors containing AAV2/5 and AAV2/9 empty capsid served as controls.

Vector production was performed following the protocol of vector core of the TarGeT Laboratory of Nates⁴⁹. Briefly, recombinant AAVs were manufactured by co-transfection of HEK293 cells and purified by cesium chloride density gradients followed by extensive dialysis against phosphate-buffered saline (PBS). Vector titers were determined by qPCR and expressed as vector genome (vg)/ml.

Animals Included in this Study

All mice experiments were approved by the local ethical committee and the “ministère de la recherche et de l′enseignement supérieur” (authorization 2016080510211993 version2). All the procedures were performed in accordance with the French regulation for the animal procedure (French decree 2013-118) and with specific European Union guidelines for the protection of animal welfare (Directive 2010/63/EU). Mice were maintained on a 12 h dark, 12 h light cycle with a humidity between 40 and 60% and an ambient temperature of 21-22° C.

### Surgery and Vector Delivery

Twelve-week-old Swiss/CD1 female mice (Janvier Labs, France) were injected into the right LG. The LG injection of AAV vectors was performed under anesthesia with a mixture of Ketamine (70 mg/kg, Imalgene® 1000, Centravet, France) and Medetomidine (1 mg/kg, Domitor®, Centravet, France). One drop of Ocry-gel (Centravet, France) was applied to each eye. The skin on the cheek under the right ear was disinfected with vetedine solution (Centravet, France) and ethanol 70% and then cut above the LG location. Next, the viral solution was injected using a 34-gauge beveled needle (Hamilton, reference 207434, Reno, NV, USA) linked to a 10-μl Hamilton syringe (1701 RN serie, Hamilton, reference 7653-01, Reno, NV, USA). Wound were closed by suture wires (Novosyn® 6/0, reference C0068006, B Braun) and then disinfected with vetedine solution (Centravet, France). After surgery, mice were treated with Buprenorphine (100 μg/kg, Brupecare®, Centravet, France) and were woken up with Atipamezole (1 mg/kg, Antisedan®, Centravet, France).

AAV vector solutions were prepared by diluting vectors at the right titer with sterile PBS and 0.01% of Fast Green (Sigma-Aldrich, reference F7252, France). For all experimental studies, mice were unilaterally injected in the right LG with 3 μl of vectors indicated below. For the transduction pattern study, AAV2/5 and AAV2/9 CAG GFP were injected at 10¹⁰ vg/LG. For the dose response study, AAV2/9 CAG-mNGF was injected at 10⁹, 10¹⁰ or 10¹¹ vg/LG. For the kinetic study, AAV2/5 CAG-mNGF was injected at 10¹⁰ vg/LG and AAV2/9 CAG-mNGF was injected at 10¹⁰ and 10¹¹ vg/LG. For the biodistribution and the biosafety studies, AAV2/9 CAG-mNGF was injected at 10¹¹ vg/LG. Animals injected with AAV2/5 or AAV2/9 empty capsid served as control.

### Corneal Abrasion

Corneal abrasions were performed as previously described^{1,3,50}. Briefly, an ocular burr (Algerbrush II, reference BR2-5 0.5 mm, Alger company) was used on mice that were anesthetized with a mixture of Ketamine (70 mg/kg, Imalgene® 1000, Centravet, France) and Medetomidine (1 mg/kg, Domitor®, Centravet, France). Abrasions were performed unilaterally. A fluorescein solution (1% in PBS, Sigma-Aldrich) was used to visualize the wound under a cobalt blue light. After abrasion, one drop of Ocry-gel (Centravet, France) was applied to each eye, mice were treated with Buprenorphine (100 μg/kg, Brupecare®, Centravet, France) and were woken up with Atipamezole (1 mg/kg, Antisedan®, Centravet, France).

### Tissue Collection and Processing

Tears were collected using a 1 μl-microcapillary (Sigma-Aldrich, reference P1424, France) for one minute, 1 day before injection and 30 days post injection for the dose response and biodistribution studies; 1 day before injection and 7, 30, 60, 120 and 180 days post injection for the kinetic and biosafety studies; 1 day before abrasion and 1, 3 and 7 days post abrasion for the corneal abrasion study.

For the transduction pattern and the biodistribution studies, mice were euthanized 30 days post injection using pentobarbital (54.7 mg/mL, 140 mg/kg, Centravet, France). They were transcardially perfused with sterile PBS and tissues were quickly dissected. Tissues were then fixed for 45 min in 4% paraformaldehyde solution (AntigenFix, Diapath, reference P0014, France) at room temperature or directly snap-frozen in liquid nitrogen and stored at −80° C. for IHC and molecular/biochemical analysis respectively.

For whole cornea imaging, mice were euthanized by cervical dislocation, enucleated with curved scissors by cutting the optic nerve. Collected eyes were then fixed for 20 min in 4% paraformaldehyde solution (AntigenFix, Diapath, reference P0014, France) at room temperature. After PBS washes, eyes were dehydrated during 2 h in 50% ethanol/PBS and then stored at 4° C. in 70% ethanol/PBS.

Tear Volume, Protein Concentration and mNGF Analyses in Tears

The volume of tears per minute (μl/min) and the protein concentration in tears (mg/ml) using the BCA protein assay kit (Fisher Scientific, reference 10678484, France) were measured and expressed as the mean±SD.

The mNGF level in tears upon a AAV2/9 CAG-mNGF injection in the LG was dosed using the mNGF ELISA kit (Sigma-Aldrich, reference RAB1119, France) following the manufacturer's recommendations. Measures were performed on a CLARIOstar microplate reader (BMG Labtech, France), and analyzed using the CLARIOstar software (version 5.60 R2). Results are expressed as the mean±SD.

### IHC Study

The following antibodies were used for IHC studies: chicken anti-GFP (Aves Labs, reference GFP-1020, 1/1000), mouse anti-Ecadh (BD Biosciences, reference 610182, 1/300), rabbit anti-Krt19 (Abcam, reference ab52625, 1/200), rabbit anti-βIII tubulin (Abcam, reference ab18207, 1/1000), goat anti-chicken Alexa Fluor 488 (Thermo Fisher Scientific, reference A-32931, 1/500), goat anti-mouse Alexa Fluor 568 (Thermo Fisher Scientific, reference A-11004, 1/500) and goat anti-rabbit Alexa Fluor 568 (Abcam, reference ab175471, 1/500), goat anti-rabbit Alexa Fluor 488 (Abcam, reference ab11008, 1/500) and goat anti-rabbit Alexa Fluor 568 (Abcam, reference ab175471, 1/500). Nuclei were counterstained with Hoechst 33342 (Thermo Fisher Scientific, reference H3570, 1/2000) and BioTracker NIR694 (Merck, reference SCT118, 1/400).

### Immunohistochemistry on LG Frozen Sections

Following fixation, LG were incubated 24 h in two successive baths of 6% and 30% sucrose and then embedded in Optimal Cutting Temperature (OCT tissue freezing medium, MM France, reference F/TFM-C) and stored at −80° C. Longitudinal sections (10 μm of thickness) were cut using a cryostat apparatus (LEICA CM3050). For primary antibodies produced in rabbit and chicken, cryosections were blocked with a mixture of 5% Goat Serum (GS, Thermo Fisher Scientific, reference 16210064), 5% of fish skin gelatin (FSG, Sigma-Aldrich, reference G7765) and 0.1% triton X-100 in PBS for 1 h at room temperature. For primary antibodies produced in mouse (mouse anti-Ecadh), the blocking step described above was followed by an incubation with goat anti-mouse immunoglobulins (Abcam, reference ab6668, 1/200) during 1 h at room temperature. Cryosections were then incubated overnight at 4° C. with primary antibodies diluted in GS/FSG/Triton/PBS mixture, washed three times with 0.1% triton X-100/PBS and subsequently incubated 1 h at room temperature with secondary antibodies diluted in GS/FSG/Triton/PBS mixture. After several PBS washes, cryosections were mounted in Fluoromount-G mounting medium (Invitrogen, reference 00-4958-02).

### Immunohistochemistry on Whole Cornea

Eyes were rehydrated in 50% ethanol/PBS during 2 h and washed twice in PBS for 15 minutes at room temperature. Corneas were dissected and permeabilized with 0.5% Triton-X-100/PBS on a rocking agitator for 1 h and then blocked in 5% GS (Thermo Fisher Scientific, reference 16210064) 2.5% FSG (Sigma-Aldrich, reference G7765) in 0.1% Triton X-100/PBS at room temperature. Corneas were incubated in primary antibody diluted in blocking solution overnight at 4° C. on a rocking agitator and rinsed in 0.1% Triton X-100/PBS at room temperature (three times 1 h). Next, samples were incubated with secondary antibodies as previously mentioned. After the washes, nuclei were stained 10 minutes with BioTracker NIR694 (Merck, reference SCT118) and washed in PBS. Corneas were cut at four corners and mounted in Fluoromount-G mounting medium (Invitrogen, reference 00-4958-02), epithelium facing the coverslip.

### Imaging

LG images were acquired using Zen Black software (version 2.3 SP1, Zeiss, France) on a LSM 880 confocal microscope (Zeiss, France). Whole LG section images were obtained using a 20×/0.8 objective while co-immunostaining of GFP with E-cadherin or Keratin19 proteins were observed via 0.36 μm step size z-stacks using a 63x/1.4 oil immersion objective. Images were then processed with Zen Black software (version 2.3 SP1, Zeiss, France) and Zen Blue lite software (version 3.2, Zeiss, France).

Whole cornea images were acquired using the navigator module on a Leica Thunder Imager Tissue microscope with Large Volume Computational Clearing (LVCC) process. Images were obtained using a 20x/0.55 objective with LAS X software (3.7.4) and processed with Imaris Bitplane software (version 9.8.0).

### Western Blot

Frozen LG were crushed with a pestle and mortar pre cooled at −80° C., solubilized in Pierce® RIPA lysis buffer (Thermo Fisher Scientific, reference 89900, France) supplemented with protease inhibitors (Halt™ Protease Inhibitor Cocktail, Thermo Fisher Scientific, reference 87786, France), homogenized on a rotating wheel at 4° C. overnight and then centrifuged at 16 900 g (Centrifuge 5418R, Eppendorf) for 30 min at 4° C. Supernatants were recovered and protein concentration of LG lysates or collected tears were quantified using the BCA protein assay kit (Thermo Fisher Scientific, reference 10678484, France). Fourty and 8 μg of proteins from LG lysates and collected tears respectively were loaded on any kD precast polyacrylamide gels (Mini-Protean® TGX™ gels, Bio Rad, reference 4568124, France). Proteins were transferred to nitrocellulose membranes (Trans-Blot Turbo Mini 0.2 μm Nitrocellulose Transfer Pack, Bio Rad, reference 1704158, France) through semi-dry transfer process (Bio Rad Trans-Blot Turbo system). Membranes were incubated with the REVERT™ total protein stain solution (LI-COR Biosciences, reference 926-11015, France) to record the overall amount of protein per well. They were then blocked for 1 h at room temperature using Intercept® blocking buffer (LI-COR Biosciences, reference 927-60001, France). They were incubated with the following primary antibodies overnight at 4° C. in LI-COR blocking buffer: chicken anti-GFP (Aves Labs, reference GFP-1020, 1/2000) or rabbit mNGF (Abcam, reference Ab52918, 1/500). Following three washes with TBS containing 0.1% Tween (TBST) for 15 min, secondary antibodies were incubated at a 1/15000 dilution in LI-COR blocking buffer: donkey anti chicken IR Dye 800CW (LI-COR Biosciences, reference 926-32218) or donkey anti rabbit IR Dye 800CW (LI-COR Biosciences, reference 926-32213). After three washes in TBST for 15 min, images were acquired with an Odyssey CLX LI-COR Imaging System (LI-COR Biosciences, France) and the quantifications were performed with Image Studio lite software (version 5.2). The GFP and the mNGF protein levels were both normalized to the total amount of protein loaded per well. The results are expressed as the mean±SD.

### AAV2/9 Biodistribution Study

LG, liver and heart from AAV2/9 CAG-mNGF-injected mice (10¹¹ vg/LG, n=7) were collected one month post injection in DNA-free, RNAse/DNAse-free and PCR inhibitor-free certified microtubes. Tissue samples were collected immediately after sacrifice, snap-frozen in liquid nitrogen and stored at −80° C. in conditions that minimize cross-contamination and avoid qPCR inhibition. Extraction of genomic DNA (gDNA) from tissues using the Gentra Puregene kit (Qiagen, reference 158445, France) and Tissue Lyser II (Qiagen, reference 85300, France) was performed in accordance with the manufacturer's recommendations. Vector genome copy number was determined using a primer/FAM-TAMRA probe combination designed to amplify a specific region of the BGH transgene. qPCR analyses were conducted on a StepOne Plus apparatus (Applied Biosystems®, Thermo Fisher Scientific, France) using 50 ng of gDNA in triplicates and the following cycling conditions: denaturation step (20 sec, 95° C.) followed by a total of 45 cycles (1 sec, 95° C.; 20 sec, 60° C.). All reactions were performed in a final volume of 20 μl containing template DNA, Premix Ex Taq (Takara/Ozyme, reference RR390L, France), 0.4 μl of ROX reference Dye (Takara/ozyme, reference RR390L, France), 0.2 μmol/L of each primer and 0.1 μmol/L of Taqman® probe (Dual-Labeled Probes, Sigma-Aldrich, France). Endogenous gDNA copy numbers were determined using the following primers/FAM-BHQ1 probe combination, designed to amplify a specific region of the murine Albumin sequence. qPCR analyses were conducted on a C1000 touch thermal cycler (Bio-Rad, France) using 50 ng of gDNA in triplicates and the following cycling conditions: denaturation step (20 sec, 95° C.) followed by a total of 45 cycles (3 sec, 95° C.; 30 sec, 60° C.). All reactions were performed in a final volume of 20 μl containing template DNA, Premix Ex Taq (Takara/Ozyme, reference RR390L,France), 0.25 μmol/L of each primer and 0.2 μmol/L of Taqman® probe (Dual-Labeled Probes, Sigma-Aldrich, France). For each sample, threshold cycle (Ct) values were compared with those obtained with different dilutions of linearized standard plasmids (containing either the BGH expression cassette or the murine Albumin gene) using Bio-Rad CFX Maestro 2.2 software (version version 5.2.008.0222) or StepOne software (version 2.3) for the C1000 touch thermal cycler and the StepOne Plus apparatus respectively. The absence of qPCR inhibition in the presence of gDNA was checked by analyzing 50 ng of gDNA extracted from tissue samples from two AAV2/9 empty capsid-injected control mice. Results were expressed in vector genome copies per diploid genome (vg/dg) as the mean±SD. The lowest limit of quantification (LLOQ) was determined as 0.001 vg/dg. Only values of vg/dg above the LLOQ were presented.

### Von Frey Test

Corneal sensitivity was evaluated using von Frey filaments (Bioseb, reference bio-VF-M) as previously described⁵¹. Filaments with define forces from 0.008 to 0.6 g were applied on the cornea of an immobilized mouse until an eye-blink reflex was observed. Mice were habituated every day for five days. Von Frey test was performed on each cornea for two consecutive days (contralateral side and injected side). The values obtained from these two days were averaged for each cornea. As the values were represented in g, we displayed them as 1/g to reflect the sensitivity. Results are expressed as the mean±SD. Behavioural experiments and analysis were performed by the same experimenter in single-blinded conditions throughout the study.

### Statistical Analysis

Data were analyzed with GraphPad Prism software (version 9.1.2, Prism, CA, USA) and expressed as the mean±SD as indicated in the Figure legends. Statistical differences between mean values were tested using Brown-Forsythe and Welch ANOVA tests followed by Dunnett's T3 multiple comparisons test, repeated measures two-way ANOVA test followed by Sidak's or Tukey's multiple comparisons test, repeated measure one-way ANOVA test followed by Dunnett's comparisons test, Friedman's one-way ANOVA test followed by Dunn's multiple comparisons test or simple linear regression as indicated in the Figure legends. Differences between values were considered significant with: * p<0.05, ** p<0.01, *** p<0.001, **** p<0.0001.

### Results

### AAV2/9 and AAV2/5 Mediate an Efficient Gene Transfer in the Lacrimal Gland

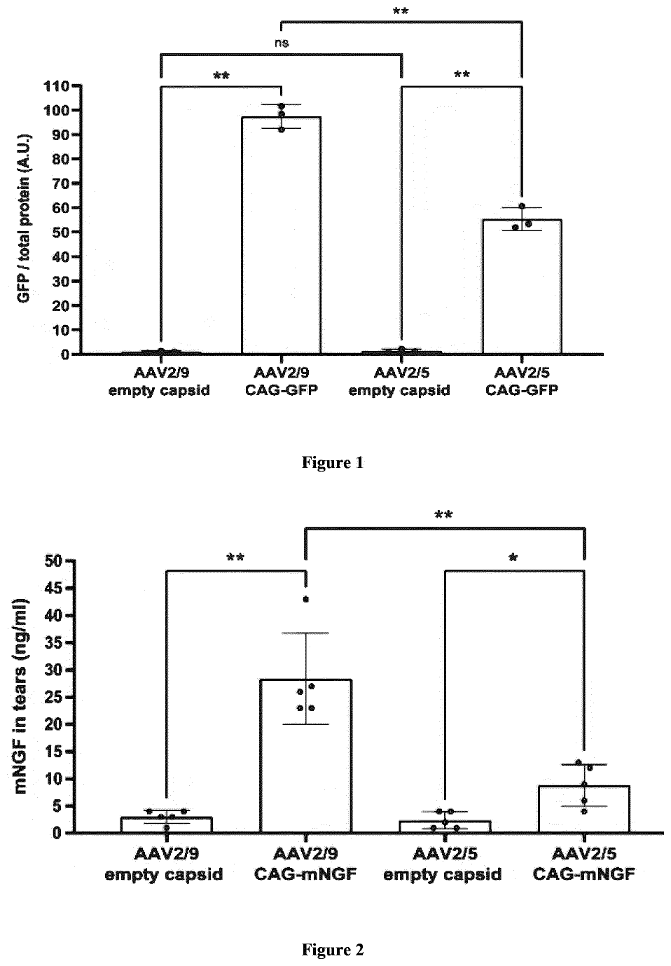

To investigate the use of the LG as bioreactor for protein secretion in the tear film, we first established an injection protocol that allows an efficient AAV-mediated gene transfer after a single injection into murine LG. We used AAV2/9-CAG-GFP and AAV2/5-CAG-GFP to monitor the extent of vector diffusion within LG. The immunostaining of GFP demonstrated that these AAV serotypes induced GFP expression in all territories of the LG (FIG. 1). To rule-out a possible tropism of AAV serotypes for a specific-cell type, we looked at GFP expression, one month after injection, in two different LG epithelial cell populations, namely the E-Cadherin (E-Cadh) positive cells, located in the acinar compartment, and the Keratin19 (Krt19) positive subpopulation, specifically localized in the ducts²⁵. The GFP/E-Cadh co-labelling showed that almost all acinar cells were GFP positive regardless of the used serotype (data not shown). Similarly, the GFP/Krt19 co-labelling demonstrated that most of the ductal cells were GFP positive after AAV2/9-CAG-GFP and AAV2/5-CAG-GFP injection (data not shown).

To evaluate a possible discrepancy between AAV serotypes 2/5 and 2/9 in the transduction efficiency, we analyzed by Western-blot the levels of GFP protein expressed in LG after injection of AAV2/9-or AAV2/5-CAG-GFP (FIG. 1). As expected, while in the LG injected with AAV2/9 or AAV2/5 empty capsids, no GFP protein was detected (data not shown), whereas high levels of GFP were detected in the LG injected with AAV2/9 or AAV2/5-CAG-GFP respectively. Interestingly, when comparing the two AAV serotypes, GFP levels were found significantly higher after AAV2/9-CAG-GFP injection than after AAV2/5-CAG-GFP injection (FIG. 1).

Taken together, our results demonstrate that LG can be widely transduced by AAV2/9 and AAV2/5, and that serotype 2/9 exhibits a higher transduction efficiency.

### The Transgene Expression Leads to the Secretion of the Resulting Protein in the Tear Film

To evaluate if AAV2/9 and AAV2/5 serotypes have a different impact on the amount of protein secreted in the tear film, we chose to express the murine nerve growth factor (mNGF) in the LG, using either AAV2/9 (AAV2/9-CAG-mNGF) or AAV2/5 (AAV2/5-CAG-mNGF). Following AAV injection into LG, the transgene expression should lead to the secretion of mNGF in the tear film. To assess this secretion, we measured mNGF level in the tear film, using ELISA and Western-blot analysis (FIG. 2). Tear film contains a basal level of mNGF²⁶, which can have two forms^{27,28}. The pro-mNGF, of higher molecular weight is cleaved to generate the mature mNGF, the active form. As expected from the GFP results, injection of AAV2/9 and AAV2/5 CAG-mNGF induced a significant increase of the mNGF levels in the tear film, whereas only endogeneous basal expression levels of mNGF were detected with the empty AAV vectors. Moreover, consistant with the GFP expression data in LG, mNGF amount in the tear film, was more than 3-fold higher after AAV2/9 injection than after AAV2/5 injection (FIG. 2). Moreover, Western blot analysis confirmed that injection of AAV2/9 and AAV2/5 CAG-mNGF led to a significant increase of the total mNGF level in the tear film (FIG. 2). Importantly, this analysis revealed that mNGF was consistently found in the tear film in its pro-mNGF form (data not shown). However, the analysis of the mature mNGF showed a maturation process (data not shown). Indeed, the mature mNGF form was close to absent from the tear film of mice injected in LG with empty AAV2/9 or AAV2/5, whereas a large amount of mature mNGF was detected after AAV-mNGF gene transfer. Notably, AAV2/9-CAG-mNGF gave rise to 4-times more of the mature form than with AAV2/5-CAG-mNGF injection (data not shown).

### AAV2/9-Mediated Gene Transfer Induces a Dose-Dependent and Long-Term Protein Secretion

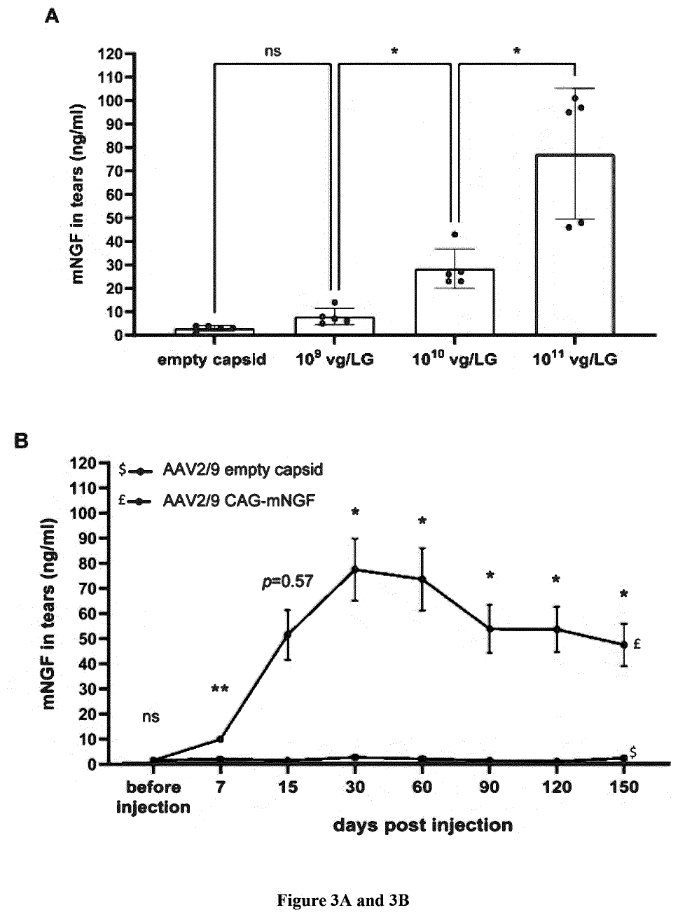

Consequently to our results indicating a better protein secretion using AAV2/9 compared to AAV2/5, we chose to focus on the serotype 2/9. To establish the optimal amount of vector genomes (vg) to be injected in LG, we tested three doses and checked the amount of secreted mNGF one month after injection (FIG. 3A). While injection of 10⁹ vg did not lead to a drastic increase of the mNGF secretion, a single injection of 10¹¹ vg induced the secretion of 75 ng of mNGF per mL of tear film. This high concentration represents a 10-time increase in comparison to physiological mNGF secretion during cornea wound healing process, where a concentration of 7.5 ng/mL was found seven days after abrasion (data not shown). We concluded that 10¹¹ vg/LG was suitable to have a significantly high increase of the protein secretion in the tear film.

Subsequently, we evaluated the dynamics of the mNGF secretion over a 6-month period (FIG. 3B). The injection of 10¹¹ vg into the LG induced a significant increase of the mNGF level found in the tear film already after a week, before reaching a peak at 30 days post injection. The mNGF secretion then decreased to reach a plateau, 120 days after injection with stable expression during the 60 following days. However, although injection of 10¹⁰ vg/LG of AAV2/9-CAG-mNGF or AAV2/5-CAG-mNG induced a peak 30 days after injection, only the serotype 2/9 induced a significant increase of the mNGF level in the tear film (data not shown). Nonetheless, the secreted mNGF level for both serotypes decreased and were no more different than the basal level. Taken together, these results show the long-lasting protein secretion when injecting 10¹¹ vg/LG, compared to 10⁹ or 10¹⁰ vg, and confirm that AAV2/9 is more efficient than AAV2/5.

AAV2/9 Vector Genome Copies in the Murine LG Correlates with the Amount of Protein Secreted in the Tear Film

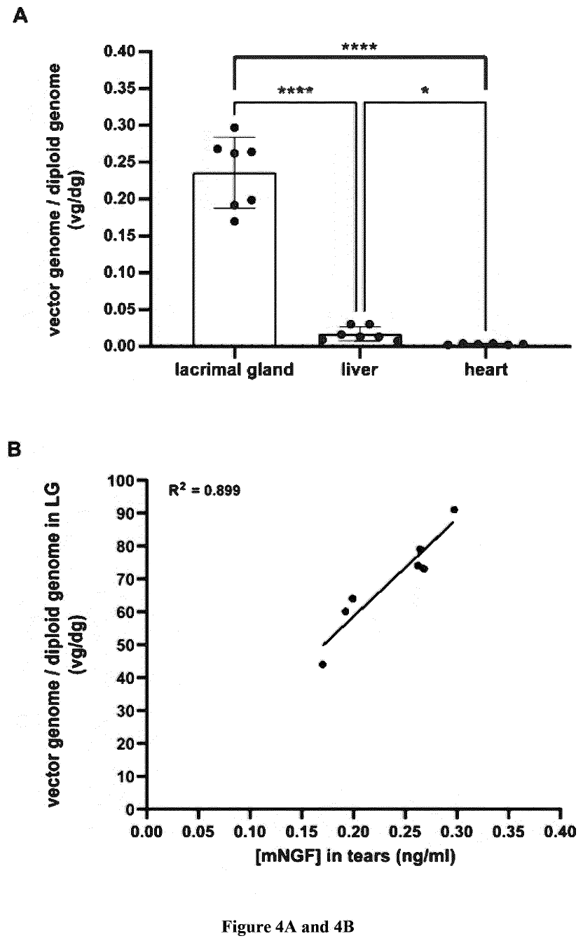

To further analyze further the use of 10¹¹ vg/LG, we investigated the biodistribution of the AAV2/9-CAG-mNGF vector after injection into LG. We measured the amount of vector genomes (vg) per diploid genome (dg) in LG, liver and heart, one month after vector injection (FIG. 4A). All injected mice showed vector genome copies in LG, indicating that the injection technique is reliable and reproducible. Interestingly, we found around 0.25 vg/dg in the LG, meaning that an average of 1 out of 4 cells was transduced with 1 copy of the AAV2/9-CAG-mNGF vector, which reflects an efficient gene transfer. Despite the detection of the AAV2/9-CAG-mNGF vector genome in liver and heart of injected mice, the levels were very low to negligeable, with 14 and 80-times less vg/dg in liver and heart respectively than in LG. Then, we compared the vg/dg values to the concentration of mNGF in the tear film of the same injected mice (FIG. 4B). Remarkably, we found a nice correlation between the vg/dg values detected in the LG and the amounts of secreted mNGF in the tears. The increase of vg/dg value corresponded to a higher mNGF secretion.

Taken together, these results confirmed the high transduction efficiency displayed by AAV2/9, which is additionally correlated with the amount of protein secreted in the tear film. Moreover, they showed a very limited distribution of AAV2/9 vector to peripheral organs after a single injection into murine LG.

AAV2/9-Mediated mNGF Secretion does not Affect LG Physiology Nor Corneal Integrity

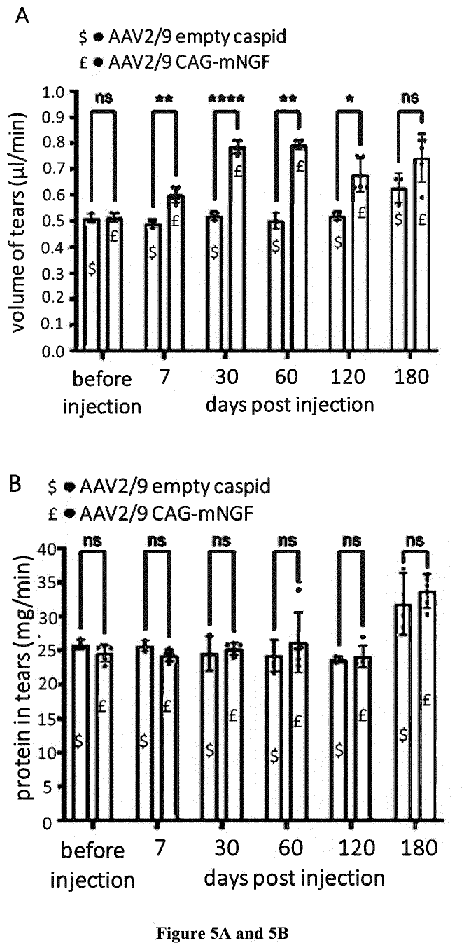

After resolving the parameters for an efficient AAV2/9-mediated mNGF gene transfer, its biodistribution, and induced mNGF secretion, we investigated the impact of the mNGF over secretion on LG physiology and corneal integrity. We injected 10¹¹ vg/LG of AAV2/9-CAG-mNGF and measured the impact on tear volume and protein concentration in tears over a period of 6 months. (FIG. 5A-B). Simultaneously to the increase of mNGF secretion we detected a significant increase of the tear volume from 7 to 120 days post-injection, when compared to the injection of AAV2/9 empty capsid (FIG. 5A). Interestingly, this increase of the tear volume was matched with a stability of the protein concentration in tears (FIG. 5B). Moreover, to rule out any impact of the injection procedure on LG physiology, we analyzed the tears before and after the AAV2/9 empty capsid injection (data not shown). Importantly, after injection of the AAV2/9 empty capsid and during the whole 6-month-period, no modification of tear volume nor of total protein concentration was observed (data not shown). This rule out any impact of the injection procedure itself on LG physiology.

In addition, we used fluorescein staining to monitor the corneal epithelium integrity and visualize any adverse effect that the injection procedure and the tear film modification could have on the cornea. Fluorescein stains areas where the corneal epithelial barrier is defected¹, as after corneal abrasion (data not shown). During the 180 days after AAV2/9 injection, we never observed any fluorescein staining, whether we injected an empty capsid, or induced mNGF over secretion. We concluded that AAV2/9 injection does not impact the corneal epithelium integrity.

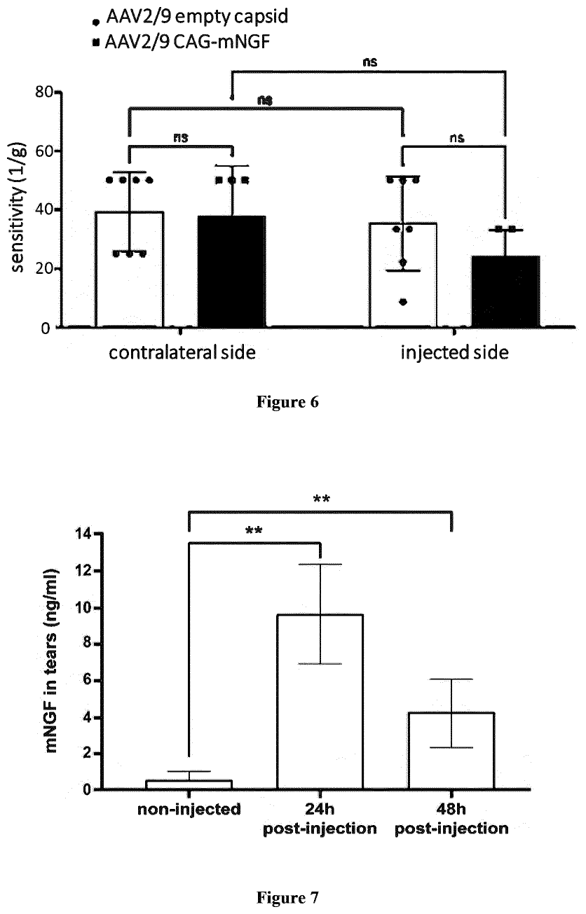

To investigate further the effect of the mNGF over secretion on cornea, we visualized corneal innervation with BIII tubulin immunolabelling, which is a pan neuronal marker. We showed that the gross morphology of corneal fibers was not affected by the over secretion of mNGF in the tear film (data not shown). Furthermore, we performed von Frey tests on the corneas of injected mice. We demonstrated that the constant over secretion of mNGF did not modify corneal sensitivity, i.e does not have deleterious effect (FIG. 6). All together, these results demonstrate that the injection procedure and mNGF over secretion respect the LG physiology and the corneal integrity.

### DISCUSSION

Among the sight threatening diseases, corneal defects are the fourth cause for blindness globally. Except for physical harm, corneal blindness often results from a combination of intrinsic and extrinsic causes. In the case of genetic diseases, such as aniridia^{29,30}, or systemic diseases, such as diabetes¹³, corneal defect is often associated with corneal microenvironment dysregulation³¹.

The aim of this study was to establish an innovative and attractive strategy to tackle corneal defects by sustainably modulating the tear film composition. For this purpose, we chose an AAV-mediated gene transfer to use LG as a bioreactor producing specific transgenes.

Nevertheless, several major variables are crucial to achieve an efficient transduction of the target cells, and thus require to be well-designed^{17,32}. First, the route of administration determines both the efficacy and the biosafety pattern of an AAV-based gene transfer. In this study, we used a local injection into LG as this organ is easily accessible by surgery. Moreover, we showed that a local injection allows concentration and time residence of AAV vectors in vicinity to the target cells, limits the biodistribution of the vector to non-targeted tissues and thus the risk of toxicity. For instance, local injection is well-established for ocular diseases, particularly in retinal disorders, as several anatomical sites are commonly targeted: subretinal, intravitreal, intracameral, suprachoroidal and topical^{33,34}. Furthermore, the injection method is a key parameter to reach a large diffusion in the target organ. While a systemic injection leads to large diffusion in all the organism including the target organ³⁵, when using local injection, the development of specific injection procedures are necessary. For example, several injections at precise coordinates are required for a large diffusion in the brain³⁶. Interestingly, a pneumatic picopump system applying multiple short-time pressure pulses has been reported to transduce the whole sciatic nerve of rodents³⁷. In this study, we used 34-gauge beveled needle linked to a 10-μL Hamilton syringe to perform injections into LG. We injected 3 μL of AAV vectors by following the path of the LG main duct. This procedure of injection led to a large diffusion in the gland, and showed an efficient gene transfer in all epithelial cell types in the murine LG. The promoter used to drive the expression of the transgene is a second parameter determining the transduction efficiency and the biosafety pattern of the injection^{17,32}. Indeed, the promoter controls the transgene expression level. Commonly, AAV-mediated gene transfer uses an ubiquitous and strong promoter, such as CAG and CMV, to achieve high transgene expression^{38,39}. Moreover, researchers usually employ such promoters when trying to establish a proof of concept for AAV-mediated gene transfer into a specific organ. In this study, we utilized a CAG promoter to provide a proof of concept of the tear fluid composition modulation after an AAV-mediated gene transfer into murine LG. On the other hand, a high transgene expression level is not always desired. For instance, transgene over expression above physiological levels has been reported to be toxic⁴⁰. This toxicity must be correlated to the serotype of the AAV vector. Indeed, the serotype represents another parameter influencing both the transduction efficiency and the biosafety of an injection as each AAV serotypes exhibit different cell and tissue tropisms⁴¹. Therefore, the combination of the route of administration, the AAV serotype, the promoter driving the transgene expression and the dose of vector have to be carefully designed as it may induce transgene expression in off-target tissues and thus lead to dramatic toxicities⁴². In this study, we compared the AAV serotypes 2/5 and 2/9 to first transduce the murine LG efficiently and then to modulate the tear film composition. Notably, while both serotypes led to an efficient gene transfer in the LG, AAV2/9 gave a better yield of GFP production and mNGF secretion compared to AAV2/5. Interestingly, although the difference of transduction efficiency was reported previously²⁴, the 2 to 4 time fold in mNGF secretion is startling. Indeed, until now, no study has reported a transgene secretion in the tear fluid after an AAV-mediated gene transfer into LG. Furthermore, we tested increasing dose of AAV2/9 to evaluate the mNGF secretion in the tear film. Interestingly, while 10¹¹ vg/LG dose led to the highest mNGF secretion, 10¹⁰ vg/LG was sufficient to induce a significant secretion. Given that we used the serotype 2/9, known to show high heart and liver tropism in rodent⁴³, and a strong CAG promoter, we performed a biodistribution study for AAV2/9. This study was paramount to determine the potential off-targets of our strategy, which may lead to both unwanted toxicity and immunogenicity. At the highest injected dose of 10¹¹ vg/LG, we showed an average of 0.25 vg/dg in LG, only 0.017 vg/dg in liver and 0.003 vg/dg in heart of injected mice, which corresponded to 14- and 80-times less vg/dg than in LG respectively. Importantly, these levels of AAV2/9 found in liver and heart were very low to negligible, from 100 to 10 000 times lower than those obtained after intravenous³⁹ or intrathecal⁴⁴ injections. Different strategies exist to limit the transgene expression in off-targets and its related adverse effects, including the use of cell-type specific promoters³⁹ or the incorporation of miRNA binding sites in the AAV gene expression cassette⁴⁵. We can speculate that the use of a 10-time lower dose of AAV2/9 vector, providing a high secretion of mNGF, could bring a lower off-target transduction. Injection of 10¹⁰ vg/LG of AAV led to a faster secretion of mNGF with AAV2/9 compared to AAV2/5. After a peak 30-day post-injection, the mNGF secretion decreased to the physiological level with both serotypes. However, injection of 10¹¹ vg/LG results in over secretion of mNGF during several months. This is an important aspect for the subsequent use of this method. While a lower dose could be used for transitory pathologies, such as corneal graft, or recurrent corneal abscesses, a higher dose could be of interest for chronical pathologies, such as neurotrophic keratitis, or dry eye diseases.

The long-term secretion of mNGF is a remarkable discovery, which must be linked to the LG physiology. As all epithelial organs, such as skin, cornea or mammary glands, the epithelial compartment contains stem cells regenerating continuously the organ, and healing it if necessary⁴⁶. The AAV vector is a non-integrative vector that can be lost when cell division occurs⁴⁷. Therefore, only non-proliferating cells will keep the transgene and lead to protein secretion. Our results demonstrate that gene therapy is successful on this epithelial organ and highlighting the low turn-over of epithelial cells in the LG. Knowing that 30 days after injection, there was around one AAV2/9 vector genome in every 4 cells, it would be of great interest to monitor over a long period of time to see which cells retain the AAV2/9 genome. This method would give a deeper understanding of LG fundamental biology. Of course, this long term follow-up could only be done at the cellular level, as with age, the LG physiology is randomly affected in individuals⁸. Therefore, measuring tear volume and tear protein content could be misleading because of aging impact more than AAV2/9 injection. Nevertheless, we demonstrated that up to 6 months after injection into the murine LG, our approach has no detrimental impact on the LG physiology.

Despite the large amount of mNGF and its constant presence on the cornea, we did not detect any impact on corneal innervation, nor on corneal sensitivity. We hypothesize that the robustness of corneal innervation maintains the system under control in physiological conditions, and mNGF alone is not sufficient to disturb this system. Most likely, under pathological conditions, i.e., physical harm, or neurotrophic keratopathy, the system would be sufficiently perturbed to visualize the effect of mNGF on corneal innervation.

Collectively, our results show that LG gene therapy could be established to modify specifically the tear film to support corneal physiology. The main challenge in using AAV vectors for epithelial organ gene therapy is the high renewal rate of epithelial cells. Here, we demonstrate that there is a non-renewing cell population in the LG that retains the secretory capabilities to produce a large amount of mNGF for over 6 months. The long term over secretion of mNGF could replace the use of NGF supplemented eyedrops, used to treat neurotrophic keratitis, such as observed in diabetes¹² or neurodegenerative diseases⁴⁸. Notably, by substituting mNGF with another gene, other corneal defects could be treated. Indeed numerous growth factor are suitable for promote healing of corneal epithelial defect (and to promote reinnervation of corneal epithelial after abrasion). Indeed local treatment of cenergermin, a recombinant human nerve growth factor, has shown higher rates of corneal healing in neurotrophic keratopathy⁵⁷. Topical VEGF-A application further promoted corneal nerve regeneration without inducing pathological neovascularization⁵⁸. PEDF has also been demonstrated to have an essential role in corneal structure and function⁵⁹.

### EXAMPLE 2: INJECTION mRNA INTO MURINE LACRIMAL GLAND

Tears were collected 24 and 48 hrs after injection of NGF mRNA in murine lacrimal gland. The tear fluid was then subjected to ELISA immunodosage. Physiologically a murine tear film contains about 0.5 ng/ml of NGF, the injection of 0.85 μg of mRNA coding for NGF leads to a transitory peak of NGF secretion in the tear, with around 9 ng/ml 24 hrs after injection, and 4 ng/ml at 48 hrs post-injection.

The Injection of mRNA in the murine lacrimal gland can lead to a transitory expression of the protein coded by the injected mRNA.

The transitory secretion of the chosen protein make this approach perfect to treat transitory defects of corneal surface.

### REFERENCES

Throughout this application, various references describe the state of the art to which this invention pertains. The disclosures of these references are hereby incorporated by reference into the present disclosure.

### REFERENCES

1. Kalha, S., Shrestha, B., Sanz Navarro, M., Jones, K. B., Klein, O. D., and Michon, F. (2018). Bmi1+Progenitor Cell Dynamics in Murine Cornea During Homeostasis and Wound Healing. Stem Cells 36, 562-573.
2. Zieske, J. D. (2004). Corneal development associated with eyelid opening. Int. J. Dev. Biol. 48, 903-911.
3. Kuony, A., Ikkala, K., Kalha, S., Magalhães, A. C., Pirttiniemi, A., and Michon, F. (2019). Ectodysplasin-A signaling is a key integrator in the lacrimal gland-cornea feedback loop. Development 146.
4. Müller, L. J., Marfurt, C. F., Kruse, F., and Tervo, T. M. T. (2003). Corneal nerves: structure, contents and function. Experimental Eye Research 76, 521-542.
5 Guier, C. P., and Stokkermans, T. J. (2022). Cornea Foreign Body Removal. In StatPearls (StatPearls Publishing).
6 Barrientez, B., Nicholas, S. E., Whelchel, A., Sharif, R., Hjortdal, J., and Karamichos, D. (2019). Corneal injury: Clinical and molecular aspects. Experimental Eye Research 186, 107709.
7. Ogawa, Y., Takeuchi, T., and Tsubota, K. (2021). Autoimmune Epithelitis and Chronic Inflammation in Sjögren's Syndrome-Related Dry Eye Disease. IJMS 22, 11820.
8. de Souza, R. G., de Paiva, C. S., and Alves, M. R. (2019). Age-related Autoimmune Changes in Lacrimal Glands. Immune Netw 19, e3.
9. Inomata, T., Sung, J., Nakamura, M., Iwagami, M., Okumura, Y., Iwata, N., Midorikawa-Inomata, A., Fujimoto, K., Eguchi, A., Nagino, K., et al. (2020). Using Medical Big Data to Develop Personalized Medicine for Dry Eye Disease. Cornea 39, S39-S46.
10. Lambiase, A., and Sacchetti, M. (2014). Diagnosis and management of neurotrophic keratitis. OPTH, 571.
11. Mastropasqua, L., Massaro-Giordano, G., Nubile, M., and Sacchetti, M. (2017). Understanding the Pathogenesis of Neurotrophic Keratitis: The Role of Corneal Nerves: THE ROLE OF CORNEAL NERVES IN NEUROTROPHIC KERATITIS. J. Cell. Physiol. 232, 717-724.
12. Markoulli, M., Flanagan, J., Tummanapalli, S. S., Wu, J., and Willcox, M. (2018). The impact of diabetes on corneal nerve morphology and ocular surface integrity. The Ocular Surface 16, 45-57.
13. Han, S. B., Yang, H. K., and Hyon, J. Y. (2018). Influence of diabetes mellitus on anterior segment of the eye. CIA Volume 14, 53-63.
14. Yeh, S.-I., Chu, T.-W., Cheng, H.-C., Wu, C.-H., and Tsao, Y.-P. (2020). The Use of Autologous Serum to Reverse Severe Contact Lens-induced Limbal Stem Cell Deficiency. Cornea 39, 736-741.
15. Kanu, L. N., and Ciolino, J. B. (2021). Nerve Growth Factor as an Ocular Therapy: Applications, Challenges, and Future Directions. Seminars in Ophthalmology 36, 224-231.
16. Eaton, A. M., Gordon, G. M., Konowal, A., Allen, A., Allen, M., Sgarlata, A., Gao, G., Wafapoor, H., and Avery, R. L. (2015). A novel eye drop application monitor to assess patient compliance with a prescribed regimen: a pilot study. Eye 29, 1383-1391.
17. Wang, D., Tai, P. W. L., and Gao, G. (2019). Adeno-associated virus vector as a platform for gene therapy delivery. Nat Rev Drug Discov 18, 358-378.
18. Mendell, J. R., Al-Zaidy, S. A., Rodino-Klapac, L. R., Goodspeed, K., Gray, S., Kay, C. N., Boye, S. L., Boye, S. E., George, L. A., Salabarria, S., et al. (2021). Current Clinical Applications of In Vivo Gene Therapy with AAVs. Molecular Therapy 29, 464-488.
19. Kuzmin, D. A., Shutova, M. V., Johnston, N. R., Smith, O. P., Fedorin, V. V., Kukushkin, Y. S., van der Loo, J. C. M., and Johnstone, E. C. (2021). The clinical landscape for AAV gene therapies. Nat Rev Drug Discov 20, 173-174.
20. Maguire, A. M., Russell, S., Chung, D. C., Yu, Z.-F., Tillman, A., Drack, A. V., Simonelli, F., Leroy, B. P., Reape, K. Z., High, K. A., et al. (2021). Durability of Voretigene Neparvovec for Biallelic RPE65-Mediated Inherited Retinal Disease. Ophthalmology 128, 1460-1468.
21. Day, J. W., Finkel, R. S., Chiriboga, C. A., Connolly, A. M., Crawford, T. O., Darras, B. T., Iannaccone, S. T., Kuntz, N. L., Peña, L. D. M., Shieh, P. B., et al. (2021). Onasemnogene abeparvovec gene therapy for symptomatic infantile-onset spinal muscular atrophy in patients with two copies of SMN2 (STRIVE): an open-label, single-arm, multicentre, phase 3 trial. The Lancet Neurology 20, 284-293.
22. Auricchio, A., Smith, A. J., and Ali, R. R. (2017). The Future Looks Brighter After 25 Years of Retinal Gene Therapy. Human Gene Therapy 28, 982-987.
23. Mohan, R. R., Martin, L. M., and Sinha, N. R. (2021). Novel insights into gene therapy in the cornea. Experimental Eye Research 202, 108361.
24. Rocha, E. M., Di Pasquale, G., Riveros, P. P., Quinn, K., Handelman, B., and Chiorini, J. A. (2011). Transduction, tropism, and biodistribution of AAV vectors in the lacrimal gland. Invest Ophthalmol Vis Sci 52, 9567-9572.
25. Kuony, A., and Michon, F. (2017). Epithelial Markers aSMA, Krt14, and Krt19 Unveil Elements of Murine Lacrimal Gland Morphogenesis and Maturation. Front Physiol 8, 739.
26. Klenkler, B., Sheardown, H., and Jones, L. (2007). Growth Factors in the Tear Film: Role in Tissue Maintenance, Wound Healing, and Ocular Pathology. The Ocular Surface 5, 228-239.
27. Bax, B., Blundell, T. L., Murray-Rust, J., and McDonald, N. Q. (1997). Structure of mouse 7S NGF: a complex of nerve growth factor with four binding proteins. Structure 5, 1275-1285.
28. Yan, R., Yalinca, H., Paoletti, F., Gobbo, F., Marchetti, L., Kuzmanic, A., Lamba, D., Gervasio, F. L., Konarev, P. V., Cattaneo, A., et al. (2019). The Structure of the Pro-domain of Mouse proNGF in Contact with the NGF Domain. Structure 27, 78-89.e3.
29. Young, T. L. (2003). Ophthalmic genetics/inherited eye disease: Current Opinion in Ophthalmology 14, 296-303.
30. Latta, L., Figueiredo, F. C., Ashery-Padan, R., Collinson, J. M., Daniels, J., Ferrari, S., Szentmáry, N., Solá, S., Shalom-Feuerstein, R., Lako, M., et al. (2021). Pathophysiology of aniridia-associated keratopathy: Developmental aspects and unanswered questions. The Ocular Surface 22, 245-266.
31. Abdul-Al, M., Kyeremeh, G. K., Saeinasab, M., Heidari Keshel, S., and Sefat, F. (2021). Stem Cell Niche Microenvironment: Review. Bioengineering 8, 108.
32. Hudry, E., and Vandenberghe, L. H. (2019). Therapeutic AAV Gene Transfer to the Nervous System: A Clinical Reality. Neuron 101, 839-862.
33. Bordet, T., and Behar-Cohen, F. (2019). Ocular gene therapies in clinical practice: viral vectors and nonviral alternatives. Drug Discovery Today 24, 1685-1693.
34. Ochakovski, G. A., Bartz-Schmidt, K. U., and Fischer, M. D. (2017). Retinal Gene Therapy: Surgical Vector Delivery in the Translation to Clinical Trials. Front. Neurosci. 11.
35. Schuster, D. J., Dykstra, J. A., Riedl, M. S., Kitto, K. F., Belur, L. R., McIvor, R. S., Elde, R. P., Fairbanks, C. A., and Vulchanova, L. (2014). Biodistribution of adeno-associated virus serotype 9 (AAV9) vector after intrathecal and intravenous delivery in mouse. Front. Neuroanat. 8.
36. Colle, M.-A., Piguet, F., Bertrand, L., Raoul, S., Bieche, I., Dubreil, L., Sloothaak, D., Bouquet, C., Moullier, P., Aubourg, P., et al. (2010). Efficient intracerebral delivery of AAV5 vector encoding human ARSA in non-human primate. Human Molecular Genetics 19, 147-158.
37. Gautier, B., Hajjar, H., Soares, S., Berthelot, J., Deck, M., Abbou, S., Campbell, G., Ceprian, M., Gonzalez, S., Fovet, C.-M., et al. (2021). AAV2/9-mediated silencing of PMP22 prevents the development of pathological features in a rat model of Charcot-Marie-Tooth disease 1 A. Nat Commun 12, 2356.
38. Powell, S. K., and Gray, S. (2015). Viral Expression Cassette Elements to Enhance Transgene Target Specificity and Expression in Gene Therapy. 15.
39. Gray, S., Foti, S. B., Schwartz, J. W., Bachaboina, L., Taylor-Blake, B., Coleman, J., Ehlers, M. D., Zylka, M. J., McCown, T. J., and Samulski, R. J. (2011). Optimizing Promoters for Recombinant Adeno-Associated Virus-Mediated Gene Expression in the Peripheral and Central Nervous System Using Self-Complementary Vectors. Human Gene Therapy 22, 1143-1153.
40. Grimm, D., Streetz, K. L., Jopling, C. L., Storm, T. A., Pandey, K., Davis, C. R., Marion, P., Salazar, F., and Kay, M. A. (2006). Fatality in mice due to oversaturation of cellular microRNA/short hairpin RNA pathways. Nature 441, 537-541.
41. Auricchio, A. (2001). Exchange of surface proteins impacts on viral vector cellular specificity and transduction characteristics: the retina as a model. Human Molecular Genetics 10, 3075-3081.
42. Hinderer, C., Katz, N., Buza, E. L., Dyer, C., Goode, T., Bell, P., Richman, L. K., and Wilson, J. M. (2018). Severe Toxicity in Nonhuman Primates and Piglets Following High-Dose Intravenous Administration of an Adeno-Associated Virus Vector Expressing Human SMN. Human Gene Therapy 29, 285-298.
43. Zincarelli, C., Soltys, S., Rengo, G., and Rabinowitz, J. E. (2008). Analysis of AAV Serotypes 1-9 Mediated Gene Expression and Tropism in Mice After Systemic Injection. Molecular Therapy 16, 1073-1080.
44. Bailey, R. M., Armao, D., Nagabhushan Kalburgi, S., and Gray, S. (2018). Development of Intrathecal AAV9 Gene Therapy for Giant Axonal Neuropathy. Molecular Therapy-Methods & Clinical Development 9, 160-171.
45. Brown, B. D., Venneri, M. A., Zingale, A., Sergi, L. S., and Naldini, L. (2006). Endogenous microRNA regulation suppresses transgene expression in hematopoietic lineages and enables stable gene transfer. Nat Med 12, 585-591.
46. Dhouailly, D. (2009). A new scenario for the evolutionary origin of hair, feather, and avian scales. Journal of Anatomy 214, 587-606.
47. Colella, P., Ronzitti, G., and Mingozzi, F. (2018). Emerging Issues in AAV-Mediated In Vivo Gene Therapy. Molecular Therapy-Methods & Clinical Development 8, 87-104.
48. Manni, L., Rocco, M. L., Bianchi, P., Soligo, M., Guaragna, M., Barbaro, S. P., and Aloe, L. (2013). Nerve growth factor: basic studies and possible therapeutic applications. Growth Factors 31, 115-122.
49. Ayuso, E., Mingozzi, F., and Bosch, F. (2010). Production, Purification and Characterization of Adeno-Associated Vectors. CGT 10, 423-436.
50. Kalha, S., Kuony, A., and Michon, F. (2018). Corneal Epithelial Abrasion with Ocular Burr As a Model for Cornea Wound Healing. J Vis Exp.
51. Fakih, D., Zhao, Z., Nicolle, P., Reboussin, E., Joubert, F., Luzu, J., Labbé, A., Rostène, W., Baudouin, C., Mélik Parsadaniantz, S., et al. (2019). Chronic dry eye induced corneal hypersensitivity, neuroinflammatory responses, and synaptic plasticity in the mouse trigeminal brainstem. J Neuroinflammation 16, 268.
52. Yamano S, Dai J, Moursi A M. Comparison of transfection efficiency of nonviral gene transfer reagents. Mol Biotechnol. 2010 November; 46 (3): 287-300.
53. Kumar P, Nagarajan A, Uchil P D. DNA Transfection Mediated by Cationic Lipid Reagents. Cold Spring Harb Protoc. 2019 Mar. 1; 2019 (3).
54. Felgner P L, Gadek T R, Holm M, Roman R, Chan H W, Wenz M, Northrop J P, Ringold G M, Danielsen M. Lipofection: a highly efficient, lipid-mediated DNA-transfection procedure. Proc Natl Acad Sci USA. 1987 November; 84 (21): 7413-7.
55. Rahimi P, Mobarakeh V I, Kamalzare S, SajadianFard F, Vahabpour R, Zabihollahi R. Comparison of transfection efficiency of polymer-based and lipid-based transfection reagents. Bratisl Lek Listy. 2018; 119 (11): 701-705.
56. Pardi N, Tuyishime S, Muramatsu H, Kariko K, Mui B L, Tam Y K, Madden T D, Hope M J, Weissman D. Expression kinetics of nucleoside-modified mRNA delivered in lipid nanoparticles to mice by various routes. J Control Release. 2015 Nov. 10; 217:345-51.
57. Pflugfelder S C, Massaro-Giordano M, Perez V L, Hamrah P, Deng S X, Espandar L, Foster C S, Affeldt J, Seedor J A, Afshari N A, Chao W, Allegretti M, Mantelli F, Dana R. Topical Recombinant Human Nerve Growth Factor (Cenegermin) for Neurotrophic Keratopathy: A Multicenter Randomized Vehicle-Controlled Pivotal Trial. Ophthalmology. 2020 January; 127 (1): 14-26.
58. Brash J T, Denti L, Ruhrberg C, Bucher F. VEGF188 promotes corneal reinnervation after injury. JCI Insight. 2019 Nov. 1; 4 (21): e130979.
59. Lee P S, Gao N, Dike M, Shkilnyy O, Me R, Zhang Y, Yu F X. Opposing Effects of Neuropilin-1 and -2 on Sensory Nerve Regeneration in Wounded Corneas: Role of Sema3C in Ameliorating Diabetic Neurotrophic Keratopathy. Diabetes. 2019 April; 68 (4): 807-818.
60. Shang Z, Li C, Liu X, Xu M, Zhang X, Li X, Barnstable C J, Zhao S, Tombran-Tink J. PEDF Gene Deletion Disrupts Corneal Innervation and Ocular Surface Function. Invest Ophthalmol Vis Sci. 2021 Jun. 1; 62 (7): 18.

1. A method for modulating tear film composition and/or increasing tear production in a subject in need thereof, comprising administering into a lacrimal gland of said subject a nucleic acid encoding a protein of interest.

2. A method for treating a corneal defects in subject in need thereof comprising administering into a lacrimal gland of said subject an effective amount of a nucleic acid encoding a protein of interest.

3. The method according to claim 2, wherein the corneal defects is caused by a condition or event selected from the group consisting of: eye surgery, a physical wound to the corneal, dry eye disease, diabetic keratopathy, a corneal thinning disorders, iridocorneal endothelial syndrome (ICE) syndrome, and neurotrophic keratitis (NK).

4. The method according to claim 3, wherein the dry eye disease is caused by Sjögren syndrome or diabetes.

5. (canceled)

6. A method for improving corneal reinnervation and/or corneal wound healing in a subject in need thereof, comprising administering into a lacrimal gland of said subject a nucleic acid encoding a protein of interest.

7. The method according to any one of claim 1, wherein the nucleic acid is a deoxyribonucleic acid.

8. The method according to any one of claim 1, wherein the nucleic acid is a messenger ribonucleic acid.

9. The method according to claim 8, wherein the messenger ribonucleic acid is administered simultaneously, separately or sequentially with a transfection reagent.

10. The method according to any one of claim 1, wherein the nucleic acid is comprised in a vector.

11. The method according to claim 10, wherein the vector is a viral vector.

12. The method according to claim 11, wherein the viral vector is an AAV vector.

13. The method according to claim 12, wherein the AAV vector is an AAV2/9 vector or AAV2/5.

14. The method according to claim 10, wherein the vector is a non-viral vector.

15. The method according to any one of claim 1, wherein the protein of interest is selected from the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), insulin-like growth factor 2 (IGF-2), bone morphogenetic protein 6 (BMP6), glial-cell derived neurotrophic factor (GDNF) and transforming growth factor (TGF).

16. The method according to claim 15, wherein the protein of interest is NGF.

17. (canceled)

18. The method according to claim 2, wherein the nucleic acid is a deoxyribonucleic acid or a messenger ribonucleic acid and is comprised in a viral vector or a non-viral vector.

19. The method according to claim 2, wherein the protein of interest is selected from the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), insulin-like growth factor 2 (IGF-2), bone morphogenetic protein 6 (BMP6), glial-cell derived neurotrophic factor (GDNF) and transforming growth factor (TGF).

20. The method according to any claim 6, wherein the nucleic acid is a deoxyribonucleic acid or a messenger ribonucleic acid and is comprised in a viral vector or a non-viral vector.

21. The method according to claim 6, wherein the protein of interest is selected from the group consisting of nerve growth factor (NGF), neuropilin 2 (NRP2), semaphorin 3C (SEMA3C), pigment epithelium-derived factor (PEDF), vascular Endothelial Growth Factor A (VEGF-A), thyroglobuline, epidermal growth factor (EGF), keratinocyte growth factor (KGF), hepatocyte growth factor (HGF), insulin-like growth factor 1 (IGF-1), insulin-like growth factor 2 (IGF-2), bone morphogenetic protein 6 (BMP6), glial-cell derived neurotrophic factor (GDNF) and transforming growth factor (TGF).
